(19) United States
(12) Patent Application Publication
(10) Pub. No.: US 2012/0214160 A1
(43) Pub. Date: Aug. 23, 2012

(54) Methods, compositions, and kits for detecting rare cells

(75) Inventors: David Deng, Mountain View, CA (US); Caifu Chen, Palo Alto, CA (US)

(73) Assignee: LIFE TECHNOLOGIES CORPORATION, Carlsbad, CA (US)

(21) Appl. No.: 13/350,764

(22) Filed: Jan. 14, 2012

Related U.S. Application Data
(60) Provisional application No. 61/433,146, filed on Jan. 14, 2011.

(51) Int. Cl. C12Q 1/68; G01N 21/64

(52) U.S. Cl. 435/6.11

(57) ABSTRACT

Disclosed herein are methods for identifying rare cells containing particular markers and/or alleles from biological samples that have not been substantially pre-processed (e.g., unprocessed whole blood). The methods described herein provide a system for digital enrichment of target cells from a biological sample and detection of such target cells, thereby allowing accurate and efficient detection and/or enumeration of such cells in the sample.

### FIELD OF THE DISCLOSURE

Disclosed herein are methods for identifying rare cells containing particular markers and/or alleles from biological samples such as blood that, optionally, have not been substantially biochemically or physically pre-processed (e.g., “unprocessed” samples). Some embodiments refer to rare target cell enrichment from mixed samples through partitioning of small sample amounts (generally referred to herein as “digital enrichment”). Some embodiments relate to the use of a highly selective method for mutation detection referred to as competitive allele-specific TaqMan PCR (“cast-PCR”). Also described are methods for diagnosing or prognosing cancer or other maladies or disorders, or efficacy of treatment for such in a subject by enriching, detecting, and analyzing individual rare cells, e.g., circulating tumor cells (CTCs), in a sample from said subject.

### BACKGROUND INFORMATION

Identification, enumeration, and characterization of rare target cells within biological fluids such as whole blood are considered by those of skill in the art to represent a critical challenge facing the medical field. For instance, statistical data suggest that only approximately 25% of cancer patients will respond to the same treatment and the frequency of circulating tumor cells (CTCs) in the blood being a key prognostic indicator. However, CTCs are very rare, with the number of approximately 1 cell in 1 milliliter of whole blood, or less than 1 CTC for every 1 billion normal blood cells. Conventional approaches are not capable of detecting target cells at such ratios. For instance, it is very difficult to detect one to two copies of target DNA/RNA (e.g., a particular allele/mutation) out of a million, or even a billion, copies of background (e.g., normal) DNA/RNA. Some methods rely upon using a large volume of the biological sample to increase the number of target cells available for analysis. The volumes required in such methods are simply impractical for routine use. Sampling a small amount from samples of a large pool to detect rare target events, even where the detection assay has enough selectivity, does not provide reliable and reproducible results due to the random sampling error of Poisson distribution. Moreover, extensive biochemical and/or mechanical enrichment processing can cause target cell losses, and are time-consuming and expensive.

Certain currently available methods may be used to some extent for processing biological samples and to detect CTCs. For example, the CellSearch™ system is currently the only identified FDA-cleared method for the enumeration of CTC in blood samples (Veridex/J&J). Using this system, it has been shown that, for certain cancers, a CTC count of greater than five cells per 7.5 milliliter of whole blood may be associated with a poor prognosis. However, the CellSearch system is based on magnetic beads coated with antibodies against the cell surface antigen EPCAM and exhibits a very low efficiency of CTC capture, especially for those CTCs with low level EPCAM expression. Thus, downstream molecular characterization of captured CTC by currently available CellSearch protocols is very difficult.

Microfluidic chips coated with capture antibodies have also been used to capture and detect CTC (e.g., systems by Massachusetts General Hospital, On-Q-ity and Biocept). The captured cells are identified with antibodies and imaged on a chip. However, microfluidic chips can only process limited amounts of blood samples (<5 mL whole blood) in a single run with limited purity. In addition, captured CTCs are difficult to release from the chips for downstream molecular characterization.

Direct reverse transcription-polymerase chain reaction (RT-PCR) gene expression analysis (e.g., cell-type specific/cancer markers) has also been used to detect CTCs in blood (e.g., systems by Adnagen GA). However, the selectivity of conventional RT-PCR is not high enough to provide reproducible and reliable results in unprocessed biological samples, and sample enrichment is typically required. Other limitations of RT-PCR systems include low CTC detection rate (10-30%) and no CTC enumeration.

Fluorescence activated cells sorting (FACS) that filter samples by cell size and/or density gradient separation area are also available. These methods have been extensively tested in academic research and some clinical research labs. However, inefficiency, low sensitivity, and cumbersome procedures are among the significant deficiencies of such systems.

There is currently no sensitive and/or specific assay available for analyzing and quantitating rare cells in biological samples without performing enrichment processes that may skew the results. As shown below, the target cell enrichment process described herein (e.g., “digital enrichment”) may be used in combination with any of a variety of detection systems to efficiently and accurately detect rare cells in biological samples. These and other advantages of the methods described herein will be apparent to the skilled artisan from the description provided herein.

### SUMMARY OF THE DISCLOSURE

Disclosed herein are methods for identifying rare target cells present in a biological sample comprising a much higher number of “normal” (e.g., non-target) cells (e.g., a high “background” and/or a low target cell to normal cell ratio). Typically, the high background of normal cells in such samples makes identifying rare target cells therein very difficult. In some embodiments, methods for identifying and enumerating rare target cells in a sample without substantially pre-processing (e.g., subjecting samples to immuno-capture, size exclusion, density gradient and/or cell sorting enrichment procedures) are provided.

In certain embodiments, the sample is compartmentalized (e.g., partitioned or separated into aliquots) to enrich rare target cells. For example, the sample may be distributed throughout multiple wells of a plate. These wells may then be subjected to one or more methods of target cell detection in parallel to provide for identification and detection of such rare cells. As such, an accurate enumeration and analysis (e.g., by molecular analysis) of rare target cells within the sample may be made. For example, within a host (e.g., a human being) having cancer, such rare target cells may be circulating tumor cells (“CTCs”) present in blood. The CTCs are present in low numbers relative to the high number of normal cells found in blood, and are therefore very difficult to detect using currently available methods. In some embodiments, the methods described herein provide for the detection of one or more such rare CTCs in a biological sample that has not been substantially pre-processed. To do so, the biological sample (e.g., unprocessed and/or untreated whole blood) may be divided into aliquots such that each aliquot contains, for example, less than five CTCs along with a higher number of normal blood cells. In certain embodiments, each aliquot will contain either zero, one, two, three, four, or five (e.g., preferably one) rare target cells (e.g., CTCs), but many more normal cells (e.g., as may be found in a normal blood sample). The aliquots (e.g., tens, hundreds or many thousands) may then be screened in parallel to identify aliquots containing rare target cell(s).

By distributing the biological sample (e.g., blood) across many aliquots, the relative ratio of target cells to normal (e.g., non-target) cells may be increased for those aliquots containing target cells. Use of a greater number of aliquots (e.g., providing a further “split” of the original sample) will typically decrease the number of normal (e.g., non-target) cells in each aliquot and serve to isolate and/or compartmentalize the target cell(s). Those aliquots containing target cell(s) will be present in those aliquots at an increased ratio of target cell(s) to non-target cells; the target cell(s) are thereby “enriched” such that improved detection of rare target cells may be achieved. The number of target cells in a biological sample may be calculated simply by counting the number of aliquots containing target cells. This process may be termed “digital enrichment” (e.g., each aliquot preferably contains either a single (1) rare target cell or zero (0) rare target cells). In some embodiments of the digital enrichment process, blood samples may be optionally diluted and/or treated as required and/or desired by the user to improve aliquot accuracy and performance.

These methods (e.g., digital enrichment) may be combined with any suitable target cell detection methods. These include, for example, methods for detecting expression of proteins and/or nucleic acids in cells. For instance, detection methods may be used to identify a cell or cells that comprise a “target nucleic acid.” The target nucleic acid may be one that has been modified by, for example, one or more mutations (e.g., a “modified target nucleic acid” or an “allelic variant”) that may be rare among normal cells. In some embodiments, then, compositions, methods and kits for identifying cells containing such allelic variations (e.g., including, but not limited to one or more single nucleotide polymorphisms (SNPs), short tandem repeats (STRs), nucleotide (NT) insertions and/or deletions) in samples comprising abundant allelic variants (e.g., wild type target nucleotide sequences) with high specificity may be combined with the digital enrichment methods. For example, the digital enrichment methods described herein may be combined with a highly selective method for mutation detection referred to as competitive allele-specific TaqMan PCR (“cast-PCR”) as described in, for example, US 2010/0221717 A1 (U.S. Ser. No. 12/641,321) and US 2010/0285478 A1 (U.S. Ser. No. 12/748,329), both of which are hereby incorporated herein by reference in their entirety into this application. Such combinations will provide an improved workflow process wherein rare target cells are first enriched (e.g., isolated or compartmentalized) and then detected using any of a variety of detection systems. As such, rare target cells may be identified and accurately quantitated from biological samples containing relatively high numbers of non-target cells.

In some embodiments, target nucleic acids (e.g., allelic variants) may be detected by analysis of ribonucleotide acid (RNA). RNA target nucleic acids may be detected directly by a suitable method, such as by reverse transcription into complementary DNA (cDNA), and detection by any suitable method(s) (e.g., using molecular beacon, TaqMan or cast-PCR methods). One advantage of assaying RNA is that a target cell typically contains many copies thereof (e.g., many copies of the target nucleic acid). In contrast, DNA may only be present in one, two, or a few copies in a target cell. Another advantage is that single-stranded RNA molecules are detected more efficiently using certain detection method(s), such as PCR. In this way, the reliable and reproducible detection of rare target cells in the background of many non-target cells is achieved.

The rare target cells may be identified by detecting in the aliquots a cell type specific marker(s) and/or one or more modified target nucleic acids (e.g., allelic variant(s)) present or at least expressed at a higher level in the rare target cells and typically not in normal cells. In some embodiments, detection of both cell type specific markers to identify target cell(s) (e.g., CTCs) using, for example, disease-related markers (e.g., abnormal fetal or cancer-related RNA, DNA, and/or protein markers)) in the sample aliquot(s), will provide additional information and confirmation of specificity and clinical or pathophysiological relevancy of target allelic variants. For example, a cancer related allelic variant detected in the same aliquot of cancer cell type specific marker(s) may assist in confirming the variant from that cell, and that it is not a random mutation from other non-target cells.

These and other embodiments, along with the advantages thereof, will be evident to the skilled artisan from the disclosure provided herein.

### BRIEF DESCRIPTION OF THE DRAWINGS

The skilled artisan will understand that the drawings described below are for illustration purposes only. The drawings are not intended to limit the scope of the present teachings in any way.

FIG. 1. Overview of exemplary digital enrichment methods.

FIG. 2. Overview of an exemplary direct CTC analysis system using digital enrichment methods combined with reverse transcription (RT) castPCR detection methods.

FIG. 3. Schematic of an illustrative embodiment of castPCR.

FIG. 4. Schematic of an illustrative embodiment of castPCR for allelic discrimination.

FIG. 5. Comparison of castPCR results using a normal blood sample (FIG. 5A) and a normal blood sample spiked with target cells (FIG. 5B).

FIG. 6. Correlation between detection of KRAS mutation and CK19 marker expression in spiked-in samples.

FIG. 7. Exemplary digital enrichment/castPCR analysis for detection of KRAS mutation in spiked-in cells.

FIG. 8. Exemplary digital enrichment/castPCR analysis for detection of EGFR mutation and CK-19 marker expression in spiked-in cells.

FIG. 9. Summary of multiple results collected on multiple days from exemplary digital enrichment/castPCR analysis for detection of KRAS and EGFR mutations in spiked-in cells.

FIG. 10. Exemplary digital enrichment/castPCR analysis for detection of CTCs in blood samples from lung cancer patients.

FIG. 11. Expression of wild type EGFR in blood samples from lung cancer patients.

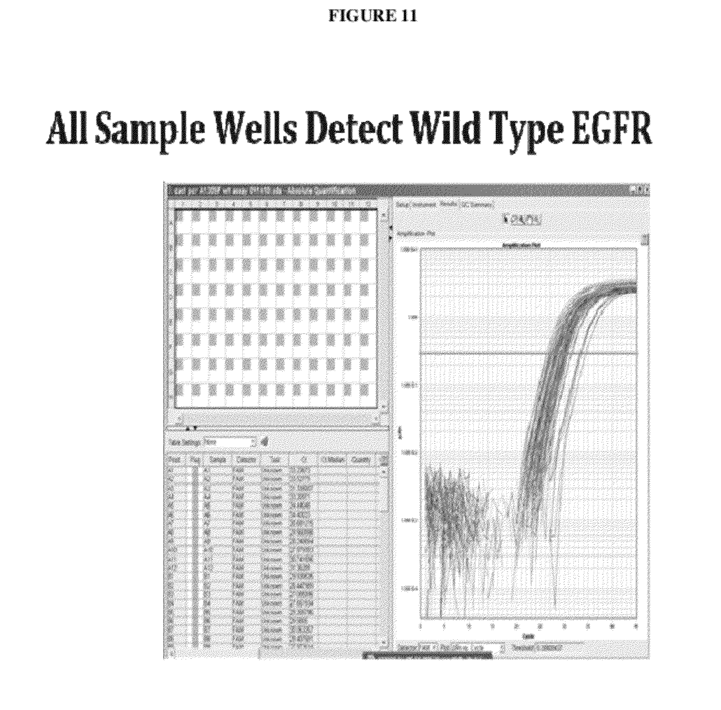

FIG. 12. Exemplary digital enrichment/castPCR analysis for detection of CTCs in blood samples from early and late stage lung cancer patients.

### DETAILED DESCRIPTION

Disclosed herein are methods for identifying rare target cells present in a biological sample comprising a much higher number of “non-target” (e.g., normal) cells (e.g., a high “background” and/or a low target cell to normal cell ratio). Typically, the high background of normal cells in such samples makes identifying rare target cells therein very difficult. In some embodiments, methods for identifying and enumerating rare target cells in a sample without substantially pre-processing the sample are provided. In certain embodiments, the sample is compartmentalized (e.g., partitioned or separated into aliquots) to enrich rare target cells. For example, the sample may be distributed throughout multiple wells of a plate. These wells may then be subjected to one or more methods of target cell detection in parallel to provide for identification and detection of such rare cells. As such, an accurate enumeration and subsequent analysis (e.g., by molecular analysis) of rare target cells within the sample may be made.

For example, within a host (e.g., a human being) having cancer, such rare target cells may be circulating tumor cells (“CTCs”) present in blood. The CTC is present in low numbers relative to the high number of normal cells found in blood, and are therefore very difficult to detect using currently available methods. In some embodiments, the methods described herein provide for the detection of one or more such rare target cells in a biological sample that has not been substantially pre-processed. To do so, the biological sample (e.g., unprocessed whole blood) may be divided into aliquots such that each aliquot contains, for example, less than five rare target cells along with a relatively large number of normal blood cells. In certain embodiments, each aliquot will contain either zero, one, two, three, four, or five rare target cells (preferably one) but many more normal cells (e.g., as may be found in a normal blood sample). The aliquots (e.g., tens, hundreds to many thousands) may then be screened in parallel to identify aliquots containing rare target cell(s). By distributing the biological sample (e.g., blood) across many aliquots, the relative ratio of target cell to normal (e.g., non-target) cells may be increased. Use of a greater number of aliquots (e.g., providing a further “split” of the original sample) will typically decrease the number of normal (e.g., non-target) cells in each aliquot and thereby improve the detection of rare target cells. In effect, rare target cells may be “enriched” using this method. The number of target cells in a biological sample may be calculated simply by counting the number of aliquots containing target cells. This process may be generally termed “digital enrichment” (e.g., an aliquot preferably contains either a single rare target cell (1) or zero rare target cells (0)). The term “digital enrichment”, however, is not limited to those embodiments in which only a single rare target cell (1) or zero rare target cells are isolated or compartmentalized, and/or present within an aliquot, but may also include embodiments in which the target cell number is, for instance, one, two, three, four, five, six, seven, eight, nine, ten (or more depending on the needs of the user). Preferably, the target cell number is five or less, and is most preferably one. In some embodiments, blood samples may be diluted (e.g., 1×, 2×, 5×, 10× or more) and/or treated as required and/or desired by the user to improve aliquot accuracy and performance (preferably without altering the overall cell number in the sample).

Biological samples containing rare cells can be obtained, for example, from any animal such as, for example, those in need of a diagnosis or prognosis or from an animal pregnant with a fetus in need of a diagnosis or prognosis. In some embodiments, a sample can be obtained from an animal suspected of being pregnant, pregnant, or that has been pregnant to detect the presence of a fetus or fetal abnormality. In another embodiment, a sample is obtained from an animal suspected of having, having, or an animal that had a disease or condition (e.g. cancer). Such a condition can be diagnosed, prognosed, or monitored, and therapy can be determined based on the methods and systems described herein.

An animal of the present invention can be a human or a domesticated animal such as a cow, chicken, pig, horse, rabbit, dog, cat, or goat. Samples derived from an animal or human can include, e.g., whole blood, sweat, tears, ear flow, sputum, lymph, bone marrow suspension, lymph, urine, saliva, semen, vaginal flow, cerebrospinal fluid, brain fluid, ascites, milk, fluid secretions of the respiratory, intestinal, or genitourinary tracts. To obtain a fluid sample (e.g., blood), any technique known in the art may be used, e.g., a syringe or other vacuum suction device.

Biological samples can also include, for example, suspended tissue samples from an animal, cell cultures or cell lines, or spiked-in cell samples.

In preferred embodiments of the disclosed invention, biological samples comprising said rare cells (including blood) are not pretreated or substantially biochemical or physical pre-processed prior to digital enrichment and subsequent analysis. In some preferred embodiments, the biological sample of interest does not undergo any cell separation or extensive manipulation prior to digital enrichment. For example, there is no separation of cells, change in cellular content, and/or redistribution of cells—e.g., such as by magnetic, affinity, or immuno-based cell separation, size exclusion, fluorescence activated cell sorting (FACS), selective lysis of a subset of the cells, and/or any other conventional enrichment methods known in the art (see, e.g., Guetta, E. M., et al., Stem Cells Dev., 13(1):93-9 (2004)) to reduce the overall number of cells and/or alter the ratio or concentration of non-target (e.g., normal) cells to target (e.g., rare) cells prior to digital enrichment (e.g., partitioning mixed samples comprising rare cells into separate aliquots).

In some embodiments, enrichment can be carried out by dispensing or pipetting small amounts of the biological sample into separate containers or vessels, and/or to distinct locations for subsequent identification, enumeration and analysis. In some embodiments, the biological sample is aliquotted into at least 2, 5, 10, 20, 50, 100, 200, 500, 1000, 5000, or 10,000 aliquots. Thus when a mixed sample comprises, for example, about 50 rare cells and is subsequently split into 50 or more different and equal aliquots, each aliquot will typically comprise 1 or 0 rare cells. In some embodiments, 5% or less, i.e., 4%, 3%, 2%, 1%, 0.5%, 0.2%, 0.1% 0.01%, 0.001%, or any other percent below 5%, preferably below 1%, of the total number of cells in one or more of the aliquots are rare cells (e.g., CTCs).

In some embodiments, the rare target cells may be identified by detecting in the aliquots a cell type specific marker(s) and/or one or more modified target nucleic acids (e.g., allelic variant(s)) present or at least expressed at a higher level in the rare target cells and typically not in normal cells. In some embodiments, detection of both cell type specific markers to identify target cell(s) (e.g., CTC) using, for example, disease-related markers (e.g., cancer-related RNA, DNA, and/or protein markers)) in the sample aliquot(s), will provide additional information and confirmation of specificity and clinical or pathophysiological relevancy of target allelic variants. For example, a cancer related allelic variant detected in the same aliquot of cancer cell type specific marker(s) may assist in confirming the variant from that cell, and that it is not a random mutation from other non-target cells.

In some embodiments, the methods described herein are used for detecting the presence of and/or quantitating the rare cells that are in a mixed sample at a concentration of less than 5%, 4%, 3%, 2%, 1%, 0.5%, 0.2%, 0.1%, 0.5%, 0.01%, 0.001%, or 0.0001% of all cells in the mixed sample, or at a ratio of less than 1:20, 1:50, 1:100, 1:200, 1:500, 1:1000, 1:2000, 1:5000, 1:10,000, 1:20,000, 1:50,000, 1:100,000, 1:200,000, 1:1,000,000, 1:2,000,000, 1:5,000,000, 1:10,000,000, 1:20,000,000, 1:50,000,000 or 1:100,000,000 of all cells in the sample, or at a concentration of less than 1, 1×10⁻¹, 1×10⁻², 1×10⁻³, 1×10⁻⁴, 1×10⁻⁵, 1×10⁻⁶, or 1×10⁻⁷ cells/μL of a fluid sample. In some embodiments, the mixed sample has as few as 500, 100, 50, 40, 30, 20, 15, 10, 9, 8, 7, 6, 5, 4, 3, 2, 1 or fewer rare cells (e.g., CTCs) per ml of sample.

These methods (i.e., digital enrichment) may be combined with any suitable target cell detection methods. These include, for example, methods for detecting expression of proteins and/or nucleic acids in cells. For instance, detection methods may be used to identify a cell or cells that comprise a “target nucleic acid.” The target nucleic acid may be one that has been modified by, for example, one or more mutations (e.g., a “modified target nucleic acid” or an “allelic variant”) that may be rare among normal cells. In some embodiments, then, compositions, methods and kits for identifying cells containing such allelic variations (e.g., including, but not limited to one or more short tandem repeats (STRs), single nucleotide polymorphisms (SNPs), nucleotide (NT) insertions and/or deletions) in samples comprising abundant allelic variants (e.g., wild type target nucleotide sequences) with high specificity may be combined with the digital enrichment methods. For example, the digital enrichment methods described herein may be combined with a highly selective method for mutation detection referred to as competitive allele-specific TaqMan PCR (“cast-PCR”) as described in, for example, US 2010/0221717 A1 (U.S. Ser. No. 12/641,321) and US 2010/0285478 A1 (U.S. Ser. No. 12/748,329), both of which are hereby incorporated herein by reference in their entirety into this application. Such combinations will provide an improved workflow process wherein rare target cells are first enriched (e.g., isolated, compartmentalized) and then detected using any of a variety of detection systems. As such, rare target cells may be identified and accurately quantitated from biological samples containing relatively high numbers of non-target (e.g., normal) cells. These embodiments and others, along with the advantages of such embodiments, will be evident to the skilled artisan from the disclosure provided herein.

The methods described herein may be used to detect rare cells, such as CTCs. The methods may also be used to modify treatment protocols for a particular disease or other condition. For instance, during chemotherapy, the number of CTCs in the blood of a patient may be monitored. In some embodiments, an increase or decrease in the number of CTCs in blood over time may indicate that the cancer treatment regimen should be altered (e.g., additional chemotherapy, a different type of chemotherapy). Similarly, the methods may be used to monitor the course of an infection by a bacterial agent and indicate whether or not a particular course of treatment is effective. For example, an increase in the number of bacterial cells in the blood may indicate that the current treatment is not effective and could suggest that treatment should be modified. Fetal cells or fetal abnormalities can also be detected and analyzed for purposes of prenatal diagnostics and screening using the disclosed inventions. Other uses for these methods would be understood by the skilled artisan, and are contemplated herein.

In some preferred embodiments, the methods described herein do not involve substantially pre-processing of the biological sample before digitally enriching and/or assaying the same for the presence of target cell(s) therein. “Pre-processing” or “substantially pre-processing” is meant to include treatment or manipulation of the biological sample such that the original cellular composition of the biological sample has been substantially altered. The methods described herein are particularly useful where the biological sample has not been pre-processed or substantially pre-processed. In some embodiments, this may mean that the biological sample is used in the form in which it was originally obtained (e.g., in cell form, as whole blood per se) and without pre-processing (or without substantial pre-processing) to isolate cells or nucleic acids therefrom. For instance, a sample may be pre-processed by subjecting the same to biochemical or physical manipulations, including, but not limited to, affinity-based separation (e.g., immuno-/antibody type, magnetic type), size-based separation or exclusion, cell lysis (e.g., apoptosis), density gradient, cell sorting enrichment procedures (e.g., flow cytometry; fluorescence activated cell sorting (FACS)) and/or any other method or procedure that alters (e.g., reduces) overall cell number of the biological sample.

In some instances, for example, whole blood may be considered not to have been substantially pre-processed where, for example, an additive such as ethylenediaminetetraacetic acid (EDTA) is introduced into the sample to prevent clotting. Similarly, in some embodiments, separation of plasma from whole blood may provide a sample that has not been substantially pre-processed so long as overall cell numbers and/or non-target to target cell ratios are not significantly altered prior to digital enrichment. Other optional treatments that may not be considered to be “substantial pre-processing” of the biological sample can also include treatment with or exposure to a stabilizer, a preservative, a fixant, an anti-apoptotic reagent, an anti-coagulation reagent, an anti-thrombotic reagent, a buffering reagent, an osmolality regulating reagent, a pH regulating reagent, or a cross-linking reagent. The biological sample can also be treated with a cell viability stain or a cell inviability stain. The skilled artisan would understand that such treatments do not substantially alter the number of cells per unit volume, and would therefore not be considered to have been “pre-processed” as the term is used herein.

When a biological sample is obtained, for example, a preservative such an anti-coagulation agent and/or a stabilizer (e.g., as in the case of blood samples) is often added to the sample prior to enrichment. This allows for extended time for analysis/detection. Thus, a sample, such as a blood sample, can be enriched and/or analyzed under any of the methods and systems herein within 1 week, 6 days, 5 days, 4 days, 3 days, 2 days, 1 day, 12 hours, 6 hours, 3 hours, 2 hours, or 1 hour from the time the sample is obtained.

Similarly, in some embodiments, a diluted sample may not be considered to have been pre-processed. For example, in some embodiments, a sample diluted 1:2, 1:3, 1:4, 1:5, 1:10, 1:20 or more may not be considered to have been substantially pre-processed since the overall cell numbers (including non-target and target cells) are not altered. Typically, however, a sample is not pre-processed if it has not been diluted, meaning that the cellular composition thereof in the aliquoted sample is representative of that present in the original biological sample (e.g., that has not been pre-processed). Thus, in some embodiments, the biological sample is separated into aliquots without additional pre-processing. Once separated into aliquots, however, the biological sample may be processed as needed to prepare the same for target cell analysis (e.g., the cell marker or allelic variant analysis methods such as by cast-PCR).

In certain embodiments, the methods may be carried out by distributing multiple aliquots of the unprocessed biological sample among discrete locations or containers such as collection vessels or tubes and/or the wells of a microtiter plate, and the like. The cells may also be directly or indirectly affixed to a solid support such as a microparticle or bead. Examples of other locations useful for separation of aliquots may include bins, sieves, pores, geometric sites, matrixes, membranes, electric traps, gaps or obstacles. The methods may also include quantitating the amount of amplification that has taken place in each of the separate locations comprising an aliquot to determine which of said aliquots contains a cell of interest.

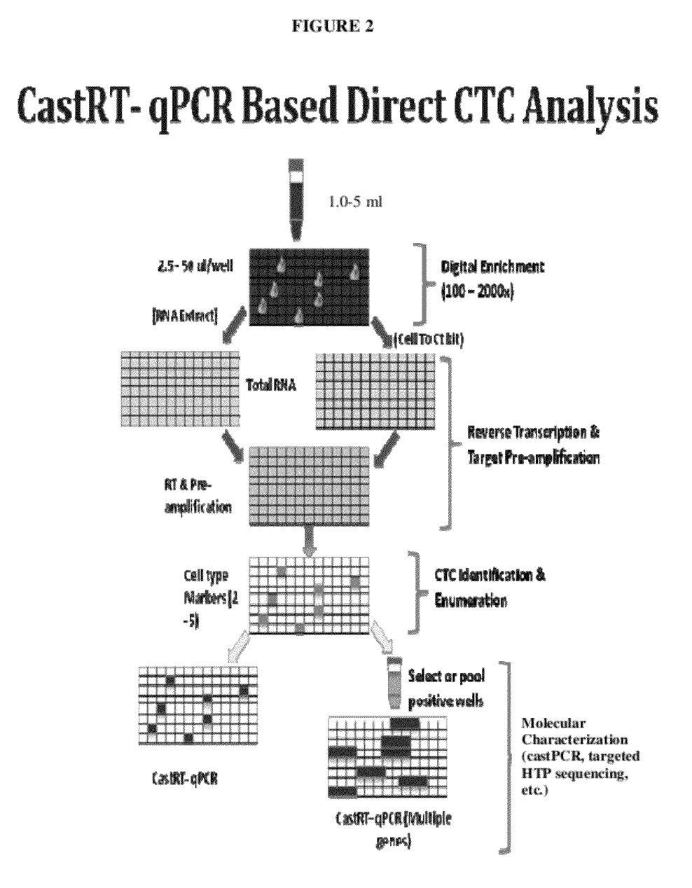

The methods described herein provide new methods for analyzing biological samples without pre-processing (or substantial pre-processing). As briefly mentioned above, potential target cells may be isolated from a biological sample that has not been substantially pre-processed and subjected to an assay for identifying allele-specific nucleotide sequences therein. Thus, these methods may comprise the steps of: 1) enrichment of target cells without substantially pre-processing the biological sample (e.g., aliquots of the same without dilution are prepared); and, 2) identification and enumeration of target cells present in the sample. The second step may be accomplished using any of the available cell and/or nucleic acid detection methods. Exemplary of such methods is the detection of allelic variants (e.g., mutations present in target cells) using PCR-based systems such as reverse transcription (RT) castPCR (FIG. 2). Other detection methods that may be used include, for example, sequencing methods, such as targeted high through-put (THTP) sequencing, allele-specific PCR (AS-PCR), proximity ligation assay (PLA) and the like. Detection methods, particularly those related to detection of nucleic acids representative of target cells, are described in more detail below. Other sensitive detection methods well-known in the art are also contemplated for use in the disclosed invention.

Target cell enrichment may be performed to isolate a very small number of target cells (e.g., 1, 2, 3, 4, 5, 6, 7, 8, 9, 10, 50, 100, or 500) directly from a biological sample. This is typically accomplished by separating target cells from one another by aliquoting the biological sample. Target cells may be considered “isolated” where other non-target cells (e.g., normal cells) are present in, for example, a portion or aliquot of the biological sample, but very few or, preferably, no other target cells are present. In some embodiments, this may be referred to as “digital enrichment” or “digital target cell enrichment” which comprises the isolation of a single target cell prior to allelic variant analysis (e.g., by castPCR). In some embodiments, target cells can be digitally enriched by 5×, 10×, 20×, 50×, 100×, 500×, 100×, 500×, 1000×, or 2000× compared to the ratio or concentration of target cells to non-target cells in the original sample using the methods disclosed herein.

Typically, the biological sample may be aliquoted to isolate target cells prior to analysis. To do so, the biological sample (e.g., unprocessed whole blood) may be divided into aliquots, wherein each aliquot contains either no target cells, or only contains one target cell (e.g., preferably no more than one) but may contain many normal cells (e.g., as may be found in a normal blood sample), that may then be screened in parallel using multiple (tens, hundreds to many thousands) sample wells to identify aliquots containing the target cell(s) (e.g., cell type specific marker(s) and/or the allelic variant(s)). Use of a greater number of aliquots (e.g., the further the original sample is split) will decrease the number of normal (e.g., non-target) cells in each aliquot which may improve the detection of rare (e.g., target) cells. By distributing the biological sample (e.g., blood) across many aliquots, the relative ratio of target cell to normal (e.g., non-target) cells is increased. In effect, target cells are enriched by this method. This aliquoting (e.g., distribution, partition, or compartmentalization) process provides aliquots containing, for example, either 1 target cell or 0 target cells, and may be referred to as “digital enrichment.” In some embodiments of digital enrichment process, blood samples can be diluted as required and/or desired by the user to improve aliquot accuracy and performance. The number of target cells in a sample may be calculated simply by counting the number of aliquots containing target cells. The number of target cells may also be identified using target cell specific markers, such as epithelial cell specific markers (e.g., cell type specific markers) and/or target cell specific mutation markers. In some embodiments, the rare (e.g., target) cells may be circulating tumor cells (CTCs).

For example, whole blood samples may be distributed into aliquots in multiple plate sample wells or tubes. Preferably, each aliquot contains a very small amount of the original sample so that, for example, each well only contains one target single cell (e.g., a pure cell) in a mix of many more (e.g., hundreds, thousands, millions, or more) non-target cells within the same aliquot. For example, given an estimated five to 50 target cells (e.g., CTCs) in a 2.5-5-ml whole blood sample, the sample may be equally divided in five to 50 μL aliquots into each well of one to five 96-well plates (e.g., 384 or 1534 wells). In this way, each aliquot may contain either zero target cells (e.g., only “background” (e.g., normal or non-cancerous) cells that do not comprise an allelic variant other than wild-type (e.g., non-mutated)) or a single target cell (e.g., a target cell, e.g., CTC, comprising an allelic variant (e.g., mutation); along with many more non-target or “background” cells (e.g., normal cells) (e.g., 1×10², 1×10³, 1×10⁴, 1×10⁵, 1×10⁶, 1×10⁷, 1×10⁸, or more)). Thus, a “digital” distribution may be achieved (e.g., either 1 or 0 target cells per aliquot). By using the methods described herein to isolate or compartmentalize target cells prior to the analysis step, an accurate target cell count in a biological sample may be determined.

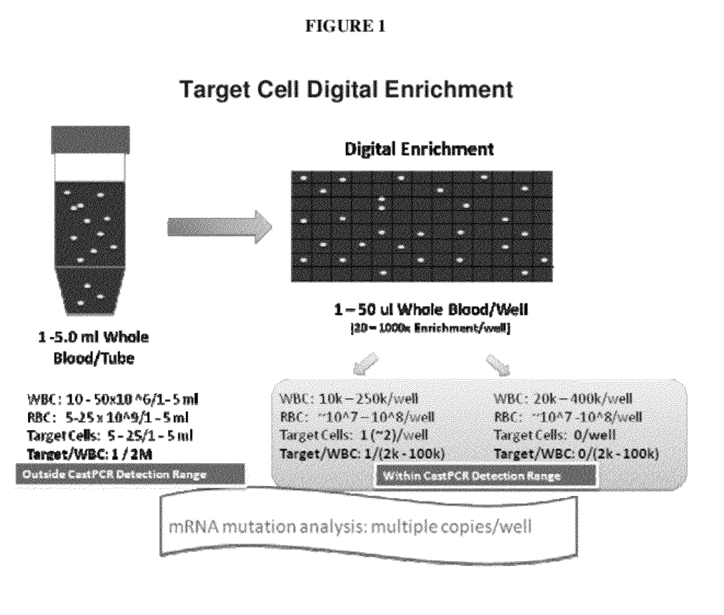

This target cell “digital enrichment” process is illustrated in FIG. 1 using human blood as an example. As described therein, one milliliter (ml) of human blood typically contains about 10⁶ white blood cells and about 5×10⁹ red blood cells. As such, the normal (e.g., non-target) cell “background” is typically very high in a blood sample. This is typically so for both proteins and nucleic acids. For example, it is known that white blood cells contain both DNA and RNA. While DNA is typically not present in red blood cells, RNA has been detected (Kabanova, et al. J. Med. Sci. 6: 156-159 (2009)). Platelets are also known to contain RNA. Thus, the nucleic acid “background” present in a normal blood sample may be quite high, making detection of nucleic acids present in and/or expressed specifically by target cells difficult and inaccurate. However, as shown in FIG. 1, digital enrichment may be performed by preparing aliquots of the blood sample to isolate and/or compartmentalize target cells prior to analysis. For instance, if one microliter (μl) of a human blood sample is aliquoted (e.g., distributed) into multiple wells (e.g., tens, hundreds, or thousands) of a microtiter plate, each well may contain about 10,000 white blood cells, 10⁷ red blood cells, and either no target cells or a single target cell (e.g., about a 1000-fold target cell enrichment). Thus, these methods may be used to detect, for instance, five or fewer target cells in one milliliter of blood (e.g., including blood that has not been substantially pre-processed). By limiting each aliquot to a single target cell, the number of target cells (e.g., CTCs) in a sample may be determined by simply counting the wells in which the target cells (e.g., allelic variant) is detected (e.g., representing those wells containing target cells).

Aliquoting biological samples “digitally” (e.g., as single cells) also provides a much improved process as compared to aliquoting target RNA/DNA after cell lysis (e.g., using conventional methods). The process allows one to determine the number of target cells in a biological sample (e.g., target cell count). This is because a single cell may contain multiple copies of target RNA molecules (e.g., of a particular allelic variant or modified target nucleic acid sequence). In certain embodiments, the method of analysis is based on RNA quantitation using, for example, detection methods such as RT-PCR. Detection of RNA provides several advantages, including, for example: 1) target mRNAs may be present in multiple copies, even thousands of copies per cell, and in CTCs, expression of target genes critical for cancer metastasis, relapse and proliferation may be much higher than normal blood cells; 2) mutations transcribed into mRNA are more likely to be functionally relevant; 3) RNA molecules are single stranded and relatively short, which potentially have better mutation detection efficiency; 4) it is possible to detect specific mutations in both RNA and DNA by assaying RNA; and, 5) in some samples (e.g., human blood), certain normal cells (e.g., red blood cells) may express less RNA than is expressed by other cells in the sample, thereby providing a lower nucleic acid background (this may also be the case for DNA in certain sample). Digital enrichment of target cells provides a powerful method for enriching target cell RNA in a sample, thereby providing for improved detection of target cells. RNAs that may be detected and thereby indicate the presence of a target cell using the methods described herein may include, for example, messenger RNA (mRNA), non-coding RNA (including long non-coding RNA), antisense RNA, CRISPR RNA, microRNA, small-interfering RNA (siRNA), pathogen-associated RNAs (e.g., bacterial or viral RNAs), and the like. Such RNA molecules may be detected using any of the available methods of detection including, for example, RT-PCR, microarray-based systems, and the like. Other advantages would be understood by one of skill in the art.

Within a host (e.g., a human being), target cells comprising allelic variations may be circulating in a biological fluid thereof. For example, certain mammals (e.g., those having cancer) may exhibit circulating tumor cells (“CTCs”) in their blood. For the purposes of this disclosure, such cells would be considered target cells. In some embodiments, the methods described herein comprise detecting one or more target cells in a biological sample that has not been substantially pre-processed (e.g., “target cell enrichment” and/or “digital enrichment”). For example, the one or more target cells may be identified within unprocessed whole blood. To do so, the biological sample (e.g., unprocessed whole blood) may be divided into aliquots, wherein each aliquot contains very few cells (e.g., less than five, preferably no more than one), which may then be screened to detect a cell of interest (e.g., the allelic variant) in one or more aliquots.

In some embodiments, the target nucleic acid may be one that has been modified by, for example, one or more mutations (e.g., a “modified target nucleic acid”), and may rarely occur (or not occur) in normal cells. As described here, such modified target nucleic acid sequences may be considered allelic variations (e.g., “allelic variants”). In some embodiments, compositions, methods and kits for identifying cells containing such allelic variations (e.g., including but not limited to one or more single nucleotide polymorphisms (SNPs), nucleotide (NT) insertions and/or deletions) in biological samples comprising abundant allelic variants (e.g., wild type target nucleotide sequences) with high specificity are provided. In particular, in some embodiments, the invention relates to a highly selective method for mutation detection referred to as competitive allele-specific TaqMan PCR (“cast-PCR”) as described in, for example, US 2010/0221717 A1 (U.S. Ser. No. 12/641,321) and US 2010/0285478 A1 (U.S. Ser. No. 12/748,329).

The allelic variants may be identified in, for example, one or more “target cells” present within a biological sample. Exemplary, non-limiting, biological samples include but are not limited to whole blood, cord blood, plasma, serum, cord serum, saliva, lymphatic fluid, cerebrospinal fluid, urine, semen, pleural fluid, milk, sweat, tears, ear flow, sputum, bone marrow, vaginal flow, brain fluid, ascites, secretions of the respiratory, intestinal, or genitourinary tracts, and the like. Allelic variants may also refer to target nucleic acids that are found in biological samples only with the occurrence of particular conditions (e.g., pregnancy, infection). Accordingly, the methods described herein may be used to identify any cell considered rare in a normal blood sample such as, for example, a fetal cell, a stem cell, and/or a bacterial cell. Other types of allelic variants that may be detected would be readily recognized by the skilled artisan and are contemplated herein.

Thus, in some embodiments, the target cell(s) may identified by detecting an allelic variant (e.g., a modified target nucleic acid sequence) therein. In these embodiments, the first allelelic variant typically represents the modified target nucleic acid sequence (e.g., mutated sequence) and the second allelic variant the unmodified (e.g., wild-type) target nucleic acid sequence. This may be accomplished by forming a reaction mixture that contains a nucleic acid sample derived from the cell of interest; a first allele-specific primer (e.g., or modified target nucleotide sequence primer) being complementary to both the first allelic variant and the second allelic variant target nucleotide acid sequence except that at least the 3′ terminal nucleotide thereof is (e.g., only) complementary to the first allelic variant; a first blocker probe being fully complementary only to the second allelic variant, “wild-type”) and comprising a 3′ non-extendable blocking moiety; a locus-specific primer complementary to the target nucleic acid at a region therein which is 3′ from and on the opposite strand to that which the first modified target nucleotide sequence primer is complementary (e.g., this primer may be complementary to both the first and second allelic variants); and, a detector probe complementary to a region of the target nucleotide sequence between that which the first modified target nucleotide sequence primer and the locus-specific primer are complementary (e.g., this primer may be complementary first and second allelic variants). An amplification reaction may then be carried out on the reaction mixture using the first modified target nucleotide sequence primer and the first locus-specific primer to form an amplicon specific for the modified target nucleic acid sequence. The amplicon (and thereby the cell of interest) may then be detected by detecting the detector probe. In some embodiments, the amount of amplicon amplified may be quantitated.

In some embodiments, a second or more additional reaction mixtures may be prepared and similarly assayed except using other primers, blocker probes, and detector probe(s) with varying specificity depending on the assay being performed. For instance, in some embodiments, a second, third, fourth or fifth (or more) modified target nucleic acid sequence may be assayed, either in the same or different aliquots. In certain embodiments, one or more of assays may also be performed to detect the unmodified target nucleic acid sequence (e.g., the wild-type or non-mutated sequence). The design of appropriate primers, blocker probes, and detector probe(s) is well within the abilities of the ordinary skilled artisan. Other variations and combinations of assays may also be used as would be understood by one of skill in the art.

CTCs may be identified using any one or more useful allele-specific and/or cell type-specific biomarkers. For example, CTCs from common solid tumors including breast, lung, prostate, colorectal, thyroid and pancreatic tissues are of predominantly epithelial cell origins. Several biomarkers for CTCs of epithelial origins are cytokeratin, EPCAM, ICAM, etc., or cancer related markers, including CEA (carcinoembryonic antigen). Similarly, prostate cancer CTCs usually express prostate-specific antigen (PSA). Allelic variants may include, for example, BRAF-1799TA, CTNNB1-121AG, CTNNB1-134CT, EGFR-2369CT, EGFR-2573TG, KRAS-34GA, KRAS-35GA, KRAS-38GA, KRAS-176CG, KRAS-183AC, NRAS-35GA, NRAS-38GA, NRAS-181CA, NRAS-183AT, TP53-524GA, TP53-637CT, TP53-721TG, TP53-733GA, TP53-742CT, TP53-743GA, TP53-817CT, and the like as described in, for example, US 2010/0221717 A1 (U.S. Ser. No. 12/641,321) and US 2010/0285478 A1 (U.S. Ser. No. 12/748,329). These markers are normally not found or expressed (or expressed in much lower level) in normal circulating blood cells. Thus, markers such as these may be used to identify CTCs, either before, simultaneously, or after assaying for the particular allele-specific marker. Additional or alternative molecular analyses of target cells may also include, for example, identifying, quantifying and/or characterizing mitochondrial DNA, telomerase, nuclear matrix proteins or microRNA.

Morphological analyses can also be used to identify target cells of interests. In some embodiments, morphological analyses can include staining rare cells and imaging the stained rare cells using bright field microscopy, e.g., to determine cell size, cell shape, nuclear size, nuclear shape, the ratio of cytoplasmic to nuclear volume, etc.

It is understood in the art that CTC count may present a reliable and independent marker for cancer prognosis for many solid tumors including breast, lung, prostate and colon. Thus, the methods described herein may be used to predict treatment outcomes and/or to monitor therapy. For instance, if the CTC count rises during treatment, the treatment may need to be adjusted to bring the CTC count down, thereby improving treatment and/or prognosis. Similarly, if the CTC count falls to zero, for instance, it may indicate that treatment could be modified and/or stopped. Other variations of these methods may be also be devised, as would be understood by one of skill in the art.

As described above, the allele of interest may be a modified target nucleic acid and, in certain cells, only one of the DNA strands contains the modification. The selective amplification of an allele of interest is often complicated by factors including the mispriming and extension of a mismatched allele-specific primer (e.g., having specificity for the modified nucleic acid) on an alternative allele (e.g., a non-modified target nucleic acid). Such mispriming and extension can be especially problematic in the detection of rare alleles (e.g., a modified target nucleic acid) present in a sample populated by an excess of another allelic variant (e.g., a non-modified target nucleic acid). When in sufficient excess, the mispriming and extension of the other allelic variant may obscure the detection of the allele of interest. When using PCR-based methods, the discrimination of a particular allele in a sample containing alternative allelic variants relies on the selective amplification of an allele of interest, while minimizing or preventing amplification of other alleles present in the sample. Certain reagents (e.g., allele-specific primers, locus-specific primers, blocking probes) may therefore be described with reference to allele, as being “allele-specific”, or the like.

A number of factors have been identified, which alone or in combination, contribute to the enhanced discriminating power of allele-specific PCR. As disclosed herein, a factor which provides a greater delta Ct value between a mismatched and matched allele-specific primer is indicative of greater discriminating power between allelic variants. Such factors found to improve discrimination of allelic variants using the present methods include, for example, the use of one or more of the following: (a) tailed allele-specific primers; (b) low allele-specific primer concentration; (c) allele-specific primers designed to have lower Tms; (d) allele-specific primers designed to target discriminating bases; (e) allele-specific blocker probes designed to prevent amplification from alternative, and potentially more abundant, allelic variants in a sample; and (f) allele-specific blocker probes and/or allele-specific primers designed to comprise modified bases in order to increase the delta Tm between matched and mismatched target sequences.

The above-mentioned factors, especially when used in combination, can influence the ability of allele-specific PCR to discriminate between different alleles present in a sample. Thus, the present disclosure relates generally to novel amplification methods referred to as cast-PCR, which utilizes a combination of factors referred to above to improve discrimination of allelic variants during PCR by increasing delta Ct values. In some embodiments, the present methods can involve high levels of selectivity, wherein one mutant molecule in a background of at least 1,000 to 1,000,000, such as about 1000-10,000, about 10,000 to 100,000, or about 100,000 to 1,000,000 wild type molecules, or any fractional ranges in between can be detected. In some embodiments, the comparison of a first set of amplicons and a second set of amplicons involving the disclosed methods provides improvements in specificity from 1,000× to 1,000,000× fold difference, such as about 1000-10,000×, about 10,000 to 100,000×, or about 100,000 to 1,000,000× fold difference, or any fractional ranges in between.

As used herein, the term “allele” refers generally to alternative DNA sequences at the same physical locus on a segment of DNA, such as, for example, on homologous chromosomes. An allele can refer to DNA sequences which differ between the same physical locus found on homologous chromosomes within a single cell or organism or which differ at the same physical locus in multiple cells or organisms (“allelelic variant”). In some instances, an allele can correspond to a single nucleotide difference at a particular physical locus. In other embodiments an allele can correspond to nucleotide (single or multiple) insertion or deletion.

As used herein, the term “allele-specific primer” refers to an oligonucleotide sequence that hybridizes to a sequence comprising an allele of interest, and which when used in PCR can be extended to effectuate first strand cDNA synthesis. Allele-specific primers are specific for a particular allele of a given target DNA or loci and can be designed to detect a difference of as little as one nucleotide in the target sequence. Allele-specific primers may comprise an allele-specific nucleotide portion, a target-specific portion, and/or a tail.

As used herein, the terms “allele-specific nucleotide portion” or “allele-specific target nucleotide” refers to a nucleotide or nucleotides in an allele-specific primer that can selectively hybridize and be extended from one allele (for example, a minor or mutant allele) at a given locus to the exclusion of the other (for example, the corresponding major or wild type allele) at the same locus.

As used herein, the term “target-specific portion” refers to the region of an allele-specific primer that hybridizes to a target polynucleotide sequence. In some embodiments, the target-specific portion of the allele-specific primer is the priming segment that is complementary to the target sequence at a priming region 5′ of the allelic variant to be detected. The target-specific portion of the allele-specific primer may comprise the allele-specific nucleotide portion. In other instances, the target-specific portion of the allele-specific primer is adjacent to the 3′ allele-specific nucleotide portion.

As used herein, the terms “tail” or “5′-tail” refers to the non-3′ end of a primer. This region typically will, although does not have to contain a sequence that is not complementary to the target polynucleotide sequence to be analyzed. The 5′ tail can be any of about 2-30, 2-5, 4-6, 5-8, 6-12, 7-15, 10-20, 15-25 or 20-30 nucleotides, or any range in between, in length.

As used herein, the term “allele-specific blocker probe” (also referred to herein as “blocker probe,” “blocker,”) refers to an oligonucleotide sequence that binds to a strand of DNA comprising a particular allelic variant which is located on the same, opposite or complementary strand as that bound by an allelic-specific primer, and reduces or prevents amplification of that particular allelic variant. As discussed in greater detail herein, allele-specific blocker probes generally comprise modifications, e.g., at the 3′-OH of the ribose ring, which prevent primer extension by a polymerase. The allele-specific blocker probe can be designed to anneal to the same or opposing strand of what the allele-specific primer anneals to and can be modified with a blocking group (e.g., a “non-extendable blocker moiety”) at its 3′ terminal end. Thus, a blocker probe can be designed, for example, so as to tightly bind to a wild type allele (e.g., abundant allelic variant) in order to suppress amplification of the wild type allele while amplification is allowed to occur on the same or opposing strand comprising a mutant allele (e.g., rare allelic variant) by extension of an allele-specific primer. In illustrative examples, the allele-specific blocker probes do not include a label, such as a fluorescent, radioactive, or chemiluminescent label

As used herein, the term “non-extendable blocker moiety” refers generally to a modification on an oligonucleotide sequence such as a probe and/or primer which renders it incapable of extension by a polymerase, for example, when hybridized to its complementary sequence in a PCR reaction. Common examples of blocker moieties include modifications of the ribose ring 3′-OH of the oligonucleotide, which prevents addition of further bases to the ′3-end of the oligonucleotide sequence a polymerase. Such 3′-OH modifications are well known in the art. (See, e.g., Josefsen, M., et al., Molecular and Cellular Probes, 23 (2009):201-223; McKinzie, P. et al., Mutagenesis. 2006, 21(6):391-7; Parsons, B. et al., Methods Mol Biol. 2005, 291:235-45; Parsons, B. et al., Nucleic Acids Res. 1992, 25:20(10):2493-6; and Morlan, J. et al., PLoS One 2009, 4 (2): e4584, the disclosures of which are incorporated herein by reference in their entireties.)

As used herein, the terms “MGB,” “MGB group,” “MGB compound,” or “MBG moiety” refers to a minor groove binder. When conjugated to the 3′ end of an oligonucleotide, an MGB group can function as a non-extendable blocker moiety.

An MGB is a molecule that binds within the minor groove of double stranded DNA. Although a general chemical formula for all known MGB compounds cannot be provided because such compounds have widely varying chemical structures, compounds which are capable of binding in the minor groove of DNA, generally speaking, have a crescent shape three dimensional structure. Most MGB moieties have a strong preference for A-T (adenine and thymine) rich regions of the B form of double stranded DNA. Nevertheless, MGB compounds which would show preference to C-G (cytosine and guanine) rich regions are also theoretically possible. Therefore, oligonucleotides comprising a radical or moiety derived from minor groove binder molecules having preference for C-G regions are also within the scope of the present invention.

Some MGBs are capable of binding within the minor groove of double stranded DNA with an association constant of 10³M⁻¹ or greater. This type of binding can be detected by well established spectrophotometric methods such as ultraviolet (UV) and nuclear magnetic resonance (NMR) spectroscopy and also by gel electrophoresis. Shifts in UV spectra upon binding of a minor groove binder molecule and NMR spectroscopy utilizing the “Nuclear Overhauser” (NOSEY) effect are particularly well known and useful techniques for this purpose. Gel electrophoresis detects binding of an MGB to double stranded DNA or fragment thereof, because upon such binding the mobility of the double stranded DNA changes.

A variety of suitable minor groove binders have been described in the literature. See, for example, Kutyavin, et al. U.S. Pat. No. 5,801,155; Wemmer, D. E., and Dervan P. B., Current Opinion in Structural Biology, 7:355-361 (1997); Walker, W. L., Kopka, J. L. and Goodsell, D. S., Biopolymers, 44:323-334 (1997); Zimmer, C.& Wahnert, U. Prog. Biophys. Molec. Bio. 47:31-112 (1986) and Reddy, B. S. P., Dondhi, S. M., and Lown, J. W., Pharmacol. Therap., 84:1-111 (1999) (the disclosures of which are herein incorporated by reference in their entireties). A preferred MGB in accordance with the present disclosure is DPI₃. Synthesis methods and/or sources for such MGBs are also well known in the art. (See, e.g., U.S. Pat. Nos. 5,801,155; 6,492,346; 6,084,102; and 6,727,356, the disclosures of which are incorporated herein by reference in their entireties.)

As used herein, the term “MGB blocker probe,” “MBG blocker,” or “MGB probe” is an oligonucleotide sequence and/or probe further attached to a minor groove binder moiety at its 3′ and/or 5′ end. Oligonucleotides conjugated to MGB moieties form extremely stable duplexes with single-stranded and double-stranded DNA targets, thus allowing shorter probes to be used for hybridization based assays. In comparison to unmodified DNA, MGB probes have higher melting temperatures (Tm) and increased specificity, especially when a mismatch is near the MGB region of the hybridized duplex. (See, e.g., Kutyavin, I. V., et al., Nucleic Acids Research, 2000, Vol. 28, No. 2: 655-661).

As used herein, the term “modified base” refers generally to any modification of a base or the chemical linkage of a base in a nucleic acid that differs in structure from that found in a naturally occurring nucleic acid. Such modifications can include changes in the chemical structures of bases or in the chemical linkage of a base in a nucleic acid, or in the backbone structure of the nucleic acid. (See, e.g., Latorra, D. et al., Hum Mut 2003, 2:79-85. Nakiandwe, J. et al., plant Method 2007, 3:2.)

As used herein, the term “detector probe” refers to any of a variety of signaling molecules indicative of amplification. For example, SYBR® Green and other DNA-binding dyes are detector probes. Some detector probes can be sequence-based (also referred to herein as “locus-specific detector probe”), for example 5′ nuclease probes. Various detector probes are known in the art, for example (TaqMan® probes described herein (See also U.S. Pat. No. 5,538,848) various stem-loop molecular beacons (See, e.g., U.S. Pat. Nos. 6,103,476 and 5,925,517 and Tyagi and Kramer, 1996, Nature Biotechnology 14:303-308), stemless or linear beacons (See, e.g., WO 99/21881), PNA Molecular Beacons™ (See, e.g., U.S. Pat. Nos. 6,355,421 and 6,593,091), linear PNA beacons (See, e.g., Kubista et al., 2001, SPIE 4264:53-58), non-FRET probes (See, e.g., U.S. Pat. No. 6,150,097), Sunrise®/Amplifluor® probes (U.S. Pat. No. 6,548,250), stem-loop and duplex Scorpion™ probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Detector probes can comprise reporter dyes such as, for example, 6-carboxyfluorescein (6-FAM) or tetrachlorofluorescin (TET). Detector probes can also comprise quencher moieties such as tetramethylrhodamine (TAMRA), Black Hole Quenchers (Biosearch), Iowa Black (IDT), QSY quencher (Molecular Probes), and Dabsyl and Dabcel sulfonate/carboxylate Quenchers (Epoch). Detector probes can also comprise two probes, wherein for example a fluor is on one probe, and a quencher on the other, wherein hybridization of the two probes together on a target quenches the signal, or wherein hybridization on a target alters the signal signature via a change in fluorescence. Detector probes can also comprise sulfonate derivatives of fluorescein dyes with SO₃ instead of the carboxylate group, phosphoramidite forms of fluorescein, phosphoramidite forms of CY5 (available, for example, from Amersham Biosciences-GE Healthcare).

As used herein, the term “locus-specific primer” refers to an oligonucleotide sequence that hybridizes to products derived from the extension of a first primer (such as an allele-specific primer) in a PCR reaction, and which can effectuate second strand cDNA synthesis of said product. Accordingly, in some embodiments, the allele-specific primer serves as a forward PCR primer and the locus-specific primer serves as a reverse PCR primer, or vice versa. In some preferred embodiments, locus-specific primers are present at a higher concentration as compared to the allele-specific primers.

As used herein, the term “rare allelic variant” refers to a target polynucleotide present at a lower level in a sample as compared to an alternative allelic variant. The rare allelic variant may also be referred to as a “minor allelic variant” and/or a “mutant allelic variant.” For instance, the rare allelic variant may be found at a frequency less than 1/10, 1/100, 1/1,000, 1/10,000, 1/100,000, 1/1,000,000, 1/10,000,000, 1/100,000,000 or 1/1,000,000,000 compared to another allelic variant for a given SNP or gene. Alternatively, the rare allelic variant can be, for example, less than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100, 000, 250, 000, 500, 000, 750,000, or 1,000,000 copies per 1, 10, 100, 1,000 micro liters of a sample or a reaction volume.

As used herein, the terms “abundant allelic variant” may refer to a target polynucleotide present at a higher level in a sample as compared to an alternative allelic variant. The abundant allelic variant may also be referred to as a “major allelic variant” and/or a “wild type allelic variant.” For instance, the abundant allelic variant may be found at a frequency greater than 10×, 100×, 1,000×, 10,000×, 100,000×, 1,000,000×, 10,000,000×, 100,000,000× or 1,000,000,000× compared to another allelic variant for a given SNP or gene. Alternatively, the abundant allelic variant can be, for example, greater than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100, 000, 250, 000, 500, 000, 750,000, 1,000,000 copies per 1, 10, 100, 1,000 micro liters of a sample or a reaction volume.

As used herein, the terms “first” and “second” are used to distinguish the components of a first reaction (e.g., a “first” reaction; a “first” allele-specific primer) and a second reaction (e.g., a “second” reaction; a “second” allele-specific primer). By convention, as used herein the first reaction amplifies a first (for example, a rare) allelic variant and the second reaction amplifies a second (for example, an abundant) allelic variant or vice versa.

As used herein, both “first allelic variant” and “second allelic variant” can pertain to alleles of a given locus from the same organism. For example, as might be the case in human samples (e.g., cells) comprising wild type alleles, some of which have been mutated to form a minor or rare allele. The first and second allelic variants of the present teachings can also refer to alleles from different organisms. For example, the first allele can be an allele of a genetically modified organism, and the second allele can be the corresponding allele of a wild type organism. The first allelic variants and second allelic variants of the present teachings can be contained on gDNA, as well as mRNA and cDNA, and generally any target nucleic acids that exhibit sequence variability due to, for example, SNP or nucleotide(s) insertion and/or deletion mutations.

As used herein, the term “thermostable” or “thermostable polymerase” refers to an enzyme that is heat stable or heat resistant and catalyzes polymerization of deoxyribonucleotides to form primer extension products that are complementary to a nucleic acid strand. Thermostable DNA polymerases useful herein are not irreversibly inactivated when subjected to elevated temperatures for the time necessary to effect destabilization of single-stranded nucleic acids or denaturation of double-stranded nucleic acids during PCR amplification. Irreversible denaturation of the enzyme refers to substantial loss of enzyme activity. Preferably a thermostable DNA polymerase will not irreversibly denature at about 90°-100° C. under conditions such as is typically required for PCR amplification.

As used herein, the term “PCR amplifying” or “PCR amplification” refers generally to cycling polymerase-mediated exponential amplification of nucleic acids employing primers that hybridize to complementary strands, as described for example in Innis et al., PCR Protocols: A Guide to Methods and Applications, Academic Press (1990). Devices have been developed that can perform thermal cycling reactions with compositions containing fluorescent indicators which are able to emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; 6,174,670; and 6,814,934 and include, but are not limited to, the ABI Prism® 7700 Sequence Detection System (Applied Biosystems, Foster City, Calif.), the ABI GeneAmp® 5700 Sequence Detection System (Applied Biosystems, Foster City, Calif.), the ABI GeneAmp® 7300 Sequence Detection System (Applied Biosystems, Foster City, Calif.), the ABI GeneAmp® 7500 Sequence Detection System (Applied Biosystems, Foster City, Calif.), the StepOne™ Real-Time PCR System (Applied Biosystems, Foster City, Calif.) and the ABI GeneAmp® 7900 Sequence Detection System (Applied Biosystems, Foster City, Calif.).

As used herein, the term “Tm′” or “melting temperature” of an oligonucleotide refers to the temperature (in degrees Celsius) at which 50% of the molecules in a population of a single-stranded oligonucleotide are hybridized to their complementary sequence and 50% of the molecules in the population are not-hybridized to said complementary sequence. The Tm of a primer or probe can be determined empirically by means of a melting curve. In some cases it can also be calculated using formulas well know in the art (See, e.g., Maniatis, T., et al., Molecular cloning: a laboratory manual/Cold Spring Harbor Laboratory, Cold Spring Harbor, N.Y.: 1982).

As used herein, the term “sensitivity” refers to the minimum amount (number of copies or mass) of a template that can be detected by a given assay. As used herein, the term “specificity” refers to the ability of an assay to distinguish between amplification from a matched template versus a mismatched template. Frequently, specificity is expressed as ΔCₜ=Ct_{mismatch}−Ct_{match}. An improvement in specificity or “specificity improvement” or “fold difference” is expressed herein as 2^{(ΔCt}^{_{—}}^{condition1-(ΔCt}^{_{—}}^{condition2)}. The term “selectivity” refers to the extent to which an AS-PCR assay can be used to determine minor (often mutant) alleles in mixtures without interferences from major (often wild type) alleles. Selectivity is often expressed as a ratio or percentage. For example, an assay that can detect 1 mutant template in the presence of 100 wild type templates is said to have a selectivity of 1:100 or 1%. As used herein, assay selectivity can also be calculated as ½^{ΔCt} or as a percentage using (½^{ΔCt}×100).

As used herein, the term “Ct” or “Ct value” refers to threshold cycle and signifies the cycle of a PCR amplification assay in which signal from a reporter that is indicative of amplicon generation (e.g., fluorescence) first becomes detectable above a background level. In some embodiments, the threshold cycle or “Ct” is the cycle number at which PCR amplification becomes exponential.

As used herein, the term “delta Ct” or “ΔCt” refers to the difference in the numerical cycle number at which the signal passes the fixed threshold between two different samples or reactions. In some embodiments delta Ct is the difference in numerical cycle number at which exponential amplification is reached between two different samples or reactions. The delta Ct can be used to identify the specificity between a matched primer to the corresponding target nucleic acid sequence and a mismatched primer to the same corresponding target nucleic acid sequence.

In some embodiments, the calculation of the delta Ct value between a mismatched primer and a matched primer is used as one measure of the discriminating power of allele-specific PCR. In general, any factor which increases the difference between the Ct value for an amplification reaction using a primer that is matched to a target sequence (e.g., a sequence comprising an allelic variant of interest) and that of a mismatched primer will result in greater allele discrimination power.

According to various embodiments, a Ct value may be determined using a derivative of a PCR curve. For example, a first, second, or nth order derivative method may be performed on a PCR curve in order to determine a Ct value. In various embodiments, a characteristic of a derivative may be used in the determination of a Ct value. Such characteristics may include, but are not limited by, a positive inflection of a second derivative, a negative inflection of a second derivative, a zero crossing of the second derivative, or a positive inflection of a first derivative. In various embodiments, a Ct value may be determined using a thresholding and baselining method. For example, an upper bound to an exponential phase of a PCR curve may be established using a derivative method, while a baseline for a PCR curve may be determined to establish a lower bound to an exponential phase of a PCR curve. From the upper and lower bound of a PCR curve, a threshold value may be established from which a Ct value is determined. Other methods for the determination of a Ct value known in the art, for example, but not limited by, various embodiments of a fit point method, and various embodiments of a sigmoidal method (See, e.g., U.S. Pat. Nos. 6,303,305; 6,503,720; 6,783,934, 7,228,237 and U.S. Application No. 2004/0096819).

In one aspect, the present invention provides compositions for use in identifying and/or quantitating an allelic variant in a nucleic acid sample. Some of these compositions can comprise: (a) an allele-specific primer; (b) an allele-specific blocker probe; (c) a detector probe; and/or (d) a locus-specific primer. In some embodiments of the compositions, the compositions may further comprise a polymerase, dNTPs, reagents and/or buffers suitable for PCR amplification, and/or a template sequence or nucleic acid sample. In some embodiments, the polymerase can be thermostable.

In another aspect, the invention provides compositions comprising: (i) a first allele-specific primer, wherein an allele-specific nucleotide portion of the first allele-specific primer is complementary to the first allelic variant of a target sequence; and (ii) a first allele-specific blocker probe that is complementary to a region of the target sequence comprising the second allelic variant, wherein said region encompasses a position corresponding to the binding position of the allele-specific nucleotide portion of the first allele-specific primer, and wherein the first allele-specific blocker probe comprises a minor groove binder.

In some illustrative embodiments, the compositions can further include a locus-specific primer that is complementary to a region of the target sequence that is 3′ from the first allelic variant and on the opposite strand.

In further embodiments, the compositions can further include a detector probe.

In another aspect, the present invention provides methods for amplifying an allele-specific sequence. Some of these methods can include: (a) hybridizing an allele-specific primer to a first nucleic acid molecule comprising a target allele; (b) hybridizing an allele-specific blocker probe to a second nucleic acid molecule comprising an alternative allele wherein the alternative allele corresponds to the same loci as the target allele; (c) hybridizing a locus-specific detector probe to the first nucleic acid molecule; (d) hybridizing a locus-specific primer to the extension product of the allele-specific primer and (e) PCR amplifying the target allele.

In another aspect, the present invention provides methods for detecting and/or quantitating an allelic variant in a mixed sample. Some of these methods can involve: (a) in a first reaction mixture hybridizing a first allele-specific primer to a first nucleic acid molecule comprising a first allele (allele-1) and in a second reaction mixture hybridizing a second allele-specific primer to a first nucleic acid molecule comprising a second allele (allele-2), wherein the allele-2 corresponds to the same loci as allele-1; (b) in the first reaction mixture hybridizing a first allele-specific blocker probe to a second nucleic acid molecule comprising allele-2 and in the second reaction mixture hybridizing a second allele-specific blocker probe to a second nucleic acid molecule comprising allele-1; (c) in the first reaction mixture, hybridizing a first detector probe to the first nucleic acid molecule and in the second reaction mixture, hybridizing a second detector probe to the first nucleic acid molecule; (d) in the first reaction mixture hybridizing a first locus-specific primer to the extension product of the first allele-specific primer and in the second reaction mixture hybridizing a second locus-specific primer to the extension product of the second allele-specific primer; and (e) PCR amplifying the first nucleic acid molecule to form a first set or sample of amplicons and PCR amplifying the second nucleic acid molecule to form a second set or sample of amplicons; and (f) comparing the first set of amplicons to the second set of amplicons to quantitate allele-1 in the sample comprising allele-2 and/or allele-2 in the sample comprising allele-1.

In yet another aspect, the present invention provides methods for detecting and/or quantitating allelic variants. Some of these methods can comprise: (a) PCR amplifying a first allelic variant in a first reaction comprising (i) a low-concentration first allele-specific primer, (ii) a first locus-specific primer, and (iii) a first blocker probe to form first amplicons; (b) PCR amplifying a second allelic variant in a second reaction comprising (i) a low-concentration second allele-specific primer, (ii) a second locus-specific primer, and (iii) a second blocker probe to form second amplicons; and (d) comparing the first amplicons to the second amplicons to quantitate the first allelic variant in the sample comprising second allelic variants.

In yet another aspect, the present invention provides methods for detecting a first allelic variant of a target sequence in a nucleic acid sample suspected of comprising at least a second allelic variant of the target sequence. Methods of this aspect include forming a first reaction mixture by combining the following: (i) a nucleic acid sample; (ii) a first allele-specific primer, wherein an allele-specific nucleotide portion of the first allele-specific primer is complementary to the first allelic variant of the target sequence; (iii) a first allele-specific blocker probe that is complementary to a region of the target sequence comprising the second allelic variant, wherein said region encompasses a position corresponding to the binding position of the allele-specific nucleotide portion of the first allele-specific primer, and wherein the first allele-specific blocker probe comprises a minor groove binder; (iv) a first locus-specific primer that is complementary to a region of the target sequence that is 3′ from the first allelic variant and on the opposite strand; and (v) a first detector probe.

Next an amplification reaction, typically a PCR amplification reaction, is carried out on the first reaction mixture using the first locus-specific primer and the first allele-specific primer to form a first amplicon. Then, the first amplicon is detected by a change in a detectable property of the first detector probe upon binding to the amplicon, thereby detecting the first allelic variant of the target gene in the nucleic acid sample. The detector probe in some illustrative embodiments is a 5′ nuclease probe. The detectable property in certain illustrative embodiments is fluorescence.

In some embodiments, the 3′ nucleotide position of the 5′ target region of the first allele-specific primer is an allele-specific nucleotide position. In certain other illustrative embodiments, including those embodiments where the 3′ nucleotide position of the 5′ target region of the first allele-specific primer is an allele-specific nucleotide position, the blocking region of the allele-specific primer encompasses the allele-specific nucleotide position. Furthermore, in illustrative embodiments, the first allele-specific blocker probe includes a minor groove binder. Furthermore, the allele-specific blocker probe in certain illustrative embodiments does not have a label, for example a fluorescent label, or a quencher.

In certain illustrative embodiments, the quantity of the first allelic variant is determined by evaluating the change in a detectable property of the first detector probe.

In certain illustrative embodiments, the method further includes forming a second reaction mixture by combining (i) the nucleic acid sample; (ii) a second allele-specific primer, wherein an allele-specific nucleotide portion of the second allele-specific primer is complementary to the second allelic variant of the target sequence; (iii) a second allele-specific blocker probe that is complementary to a region of the target sequence comprising the first allelic variant, wherein said region encompasses a position corresponding to the binding position of the allele-specific nucleotide portion of the second allele-specific primer, and wherein the second allele-specific blocker probe comprises a minor groove binder; (iv) a second locus-specific primer that is complementary to a region of the target sequence that is 3′ from the second allelic variant and on the opposite strand; and (v) a second detector probe. Next, an amplification reaction is carried out on the second reaction mixture using the second allele-specific primer and the locus-specific primer, to form a second amplicon. Then the second amplicon is detected by a change in a detectable property of the detector probe.

In certain embodiments, the method further includes comparing the change in a detectable property of the first detector probe in the first reaction mixture to the change in a detectable property of the second detector probe in the second reaction mixture.

In yet another aspect, the present invention provides a reaction mixture that includes the following (i) nucleic acid molecule; (ii) an allele-specific primer, wherein an allele-specific nucleotide portion of the allele-specific primer is complementary to a first allelic variant of a target sequence; (iii) an allele-specific blocker probe that is complementary to a region of the target sequence comprising a second allelic variant, wherein said region encompasses a position corresponding to the binding position of the allele-specific nucleotide portion of the allele-specific primer, and wherein the allele-specific blocker probe comprises a minor groove binder; (iv) a locus-specific primer that is complementary to a region of the target sequence that is 3′ from the first allelic variant and on the opposite strand; and (v) a detector probe.

In certain embodiments, the methods of the invention are used to detect a first allelic variant that is present at a frequency less than 1/10, 1/100, 1/1,000, 1/10,000, 1/100,000, 1/1,000,000, 1/10,000,000, 1/100,000,000 or 1/1,000,000,000, and any fractional ranges in between, of a second allelic variant for a given SNP or gene. In other embodiments, the methods are used to detect a first allelic variant that is present in less than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100, 000, 250, 000, 500, 000, 750,000, 1,000,000 copies per 1, 10, 100, 1,000 micro liters, and any fractional ranges in between, of a sample or a reaction volume.

In some embodiments the first allelic variant is a mutant. In some embodiments the second allelic variant is wild type. In some embodiments, the present methods can involve detecting one mutant molecule in a background of at least 1,000 to 1,000,000, such as about 1000 to 10,000, about 10,000 to 100,000, or about 100,000 to 1,000,000 wild type molecules, or any fractional ranges in between. In some embodiments, the methods can provide high sensitivity and the efficiency at least comparable to that of TaqMan®-based assays.

In some embodiments, the comparison of the first amplicons and the second amplicons involving the disclosed methods provides improvements in specificity from 1,000× to 1,000,000× fold difference, such as about 1000 to 10,000×, about 10,000 to 100,000×, or about 100,000 to 1,000,000× fold difference, or any fractional ranges in between. In some embodiments, the size of the amplicons range from about 60-120 nucleotides long.

In another aspect, the present invention provides kits for quantitating a first allelic variant in a sample comprising an alternative second allelic variants that include: (a) a first allele-specific primer; (b) a second allele-specific primer; (c), a first locus-specific primer; (d) a second locus-specific primer; (e) a first allele-specific blocker probe; (f) a second allele-specific blocker probe; and (g) a polymerase. In some embodiments of the disclosed kits, the kit further comprises a first locus-specific detector probe and a second locus-specific detector probe.

In another aspect, the present invention provides kits that include two or more containers comprising the following components independently distributed in one of the two or more containers: (i) a first allele-specific primer, wherein an allele-specific nucleotide portion of the first allele-specific primer is complementary to the first allelic variant of a target sequence; and (ii) a first allele-specific blocker probe that is complementary to a region of the target sequence comprising the second allelic variant, wherein said region encompasses a position corresponding to the binding position of the allele-specific nucleotide portion of the first allele-specific primer, and wherein the first allele-specific blocker probe comprises a minor groove binder.

In some illustrative embodiments, the kits can further include a locus-specific primer that is complementary to a region of the target sequence that is 3′ from the first allelic variant and on the opposite strand.

In other embodiments, the kits can further include a detector probe.

In some embodiments, the compositions, methods, and/or kits can be used in detecting circulating cells in diagnosis. In one embodiment, the compositions, methods, and/or kits can be used to detect tumor cells in blood for early cancer diagnosis. In some embodiments, the compositions, methods, and/or kits can be used for cancer or disease-associated genetic variation or somatic mutation detection and validation. In some embodiments, the compositions, methods, and/or kits can be used for genotyping tera-, tri- and di-allelic SNPs. In some embodiments, the compositions, methods, and/or kits can be used for DNA typing from mixed DNA samples for QC and human identification assays, cell line QC for cell contaminations, allelic gene expression analysis, virus typing/rare pathogen detection, mutation detection from pooled samples, detection of circulating tumor cells in blood, and/or prenatal diagnostics.

In some embodiments, the compositions, methods, and/or kits are compatible with various instruments such as, for example, SDS instruments from Applied Biosystems (Foster City, Calif.).

Allele-specific primers (ASPs) designed with low Tms exhibit increased discrimination of allelic variants. In some embodiments, the allele-specific primers are short oligomers ranging from about 15-30, such as about 16-28, about 17-26, about 18-24, or about 20-22, or any range in between, nucleotides in length. In some embodiments, the Tm of the allele-specific primers range from about 50° C. to 70° C., such as about 52° C. to 68° C. (e.g., 53° C.), about 54° C. to 66° C., about 56° C. to 64° C., about 58° C. to 62° C., or any range in between. In other embodiments, the Tm of the allele-specific primers is about 3° C. to 6° C. higher than the anneal/extend temperature of the PCR cycling conditions employed during amplification.

Low allele-specific primer concentration can also improve selectivity. Reduction in concentration of allele-specific primers below 900 nM can increase the delta Ct between matched and mismatched sequences. In some embodiments of the disclosed compositions, the concentration of allele-specific primers ranges from about 20 nM to 900 nM, such as about 50 nM to 700 nM, about 100 nM to 500 nM, about 200 nM to 300 nM, about 400 nM to 500 nM, or any range in between. In some exemplary embodiments, the concentration of the allele-specific primers is between about 200 nM to 400 nM.

In some embodiments, allele-specific primers can comprise an allele-specific nucleotide portion that is specific to the target allele of interest. The allele-specific nucleotide portion of an allele-specific primer is complementary to one allele of a gene, but not another allele of the gene. In other words, the allele-specific nucleotide portion binds to one or more variable nucleotide positions of a gene that is nucleotide positions that are known to include different nucleotides for different allelic variants of a gene. The allele-specific nucleotide portion is at least one nucleotide in length. In exemplary embodiments, the allele-specific nucleotide portion is one nucleotide in length. In some embodiments, the allele-specific nucleotide portion of an allele-specific primer is located at the 3′ terminus of the allele-specific primer. In other embodiments, the allele-specific nucleotide portion is located about 1-2, 3-4, 5-6, 7-8, 9-11, 12-15, or 16-20 nucleotides in from the 3′ most-end of the allele-specific primer.

Allele-specific primers designed to target discriminating bases can also improve discrimination of allelic variants. In some embodiments, the nucleotide of the allele-specific nucleotide portion targets a highly discriminating base (e.g., for detection of A/A, A/G, G/A, G/G, A/C, or C/A alleles). Less discriminating bases, for example, may involve detection of C/C, T/C, G/T, T/G, C/T alleles. In some embodiments, for example when the allele to be detected involves A/G or C/T SNPs, A or G may be used as the 3′ allele-specific nucleotide portion of the allele-specific primer (e.g., if A/T is the target allele), or C or T may be used as the 3′ allele-specific nucleotide portion of the allele-specific primer (e.g., if C/G is the target allele). In other embodiments, A may be used as the nucleotide-specific portion at the 3′ end of the allele specific primer (e.g., the allele-specific nucleotide portion) when detecting and/or quantifying A/T SNPs. In other embodiments, G may be used as the nucleotide-specific portion at the 3′ end of the allele specific primer when detecting and/or quantifying C/G SNPs.

In some embodiments, the allele-specific primer can comprise a target-specific portion that is specific to the polynucleotide sequence (or locus) of interest. In some embodiments the target-specific portion is about 75-85%, 85-95%, 95-99% or 100% complementary to the target polynucleotide sequence of interest. In some embodiments, the target-specific portion of the allele-specific primer can comprise the allele-specific nucleotide portion. In other embodiments, the target-specific portion is located 5′ to the allele-specific nucleotide portion. The target-specific portion can be about 4-30, about 5-25, about 6-20, about 7-15, or about 8-10 nucleotides in length. In some embodiments, the Tm of the target specific portion is about 5° C. below the anneal/extend temperature used for PCR cycling. In some embodiments, the Tm of the target specific portion of the allele-specific primer ranges from about 51° C. to 60° C., about 52° C. to 59° C., about 53° C. to 58° C., about 54° C. to 57° C., about 55° C. to 56° C., or about 50° C. to about 60° C.

In some embodiments of the disclosed methods and kits, the target-specific portion of the first allele-specific primer and the target-specific portion of the second allele-specific primer comprise the same sequence. In other embodiments, the target-specific portion of the first allele-specific primer and the target-specific portion of the second allele-specific primer are the same sequence.

In some embodiments, the allele-specific primer comprises a tail. Allele-specific primers comprising tails, enable the overall length of the primer to be reduced, thereby lowering the Tm without significant impact on assay sensitivity. In some exemplary embodiments, the tail is on the 5′ terminus of the allele-specific primer. In some embodiments, the tail is located 5′ of the target-specific portion and/or allele-specific nucleotide portion of the allele-specific primer. In some embodiments, the tail is about 65-75%, about 75-85%, about 85-95%, about 95-99% or about 100% non-complementary to the target polynucleotide sequence of interest. In some embodiments the tail can be about 2-40, such as about 4-30, about 5-25, about 6-20, about 7-15, or about 8-10 nucleotides in length. In some embodiments the tail is GC-rich. For example, in some embodiments the tail sequence is comprised of about 50-100%, about 60-100%, about 70-100%, about 80-100%, about 90-100% or about 95-100% G and/or C nucleotides. The tail of the allele-specific primer may be configured in a number of different ways, including, but not limited to a configuration whereby the tail region is available after primer extension to hybridize to a complementary sequence (if present) in a primer extension product. Thus, for example, the tail of the allele-specific primer can hybridize to the complementary sequence in an extension product resulting from extension of a locus-specific primer. In some embodiments of the disclosed methods and kits, the tail of the first allele-specific primer and the tail of the second allele-specific primer comprise the same sequence. In other embodiments, the 5′ tail of the first allele-specific primer and the 5′ tail of the second allele-specific primer are the same sequence.

Allele-specific blocker probes (or ASBs) (herein sometimes referred to as “blocker probes”) may be designed as short oligomers that are single-stranded and have a length of 100 nucleotides or less, more preferably 50 nucleotides or less, still more preferably 30 nucleotides or less and most preferably 20 nucleotides or less with a lower limit being approximately 5 nucleotides. In some embodiments, the Tm of the blocker probes range from 60° C. to 70° C., 61° C. to 69° C., 62° C. to 68° C., 63° C. to 67° C., 64° C. to 66° C., or about 60° C. to about 63° C., or any range in between. In yet other embodiments, the Tm of the allele-specific blocker probes is about 3° C. to 6° C. higher than the anneal/extend temperature in the PCR cycling conditions employed during amplification.

In some embodiments, the blocker probes are not cleaved during PCR amplification. In some embodiments, the blocker probes comprise a non-extendable blocker moiety at their 3′-ends. In some embodiments, the blocker probes can further comprise other moieties (including, but not limited to additional non-extendable blocker moieties, quencher moieties, fluorescent moieties, etc) at their 3′-end, 5′-end, and/or any internal position in between. In some embodiments, the allele position is located about 5-15, such as about 5-11, about 6-10, about 7-9, about 7-12, or about 9-11, such as about 6, about 7, about 8, about 9, about 10, or about 11 nucleotides away from the non-extendable blocker moiety of the allele-specific blocker probes when hybridized to their target sequences. In some embodiments, the non-extendable blocker moiety can be, but is not limited to, an amine (NH₂), biotin, PEG, DPI₃, or PO₄. In some preferred embodiments, the blocker moiety is a minor groove binder (MGB) moiety. (The oligonucleotide-MGB conjugates of the present invention are hereinafter sometimes referred to as “MGB blocker probes” or “MGB blockers.”)

As disclosed herein, the use of MGB moieties in allele-specific blocker probes can increase the specificity of allele-specific PCR. One possibility for this effect is that, due to their strong affinity to hybridize and strongly bind to complementary sequences of single or double stranded nucleic acids, MGBs can lower the Tm of linked oligonucleotides (see, for example, Kutyavin, I., et al., Nucleic Acids Res., 2000, Vol. 28, No. 2: 655-661). Oligonucleotides comprising MGB moieties have strict geometric requirements since the linker between the oligonucleotide and the MGB moiety must be flexible enough to allow positioning of the MGB in the minor groove after DNA duplex formation. Thus, MGB blocker probes can provide larger Tm differences between matched versus mismatched alleles as compared to conventional DNA blocker probes.

In general, MGB moieties are molecules that bind within the minor groove of double stranded DNA. Although a generic chemical formula for all known MGB compounds cannot be provided because such compounds have widely varying chemical structures, compounds which are capable of binding in the minor groove of DNA, generally speaking, have a crescent shape three dimensional structure. Most MGB moieties have a strong preference for A-T (adenine and thymine) rich regions of the B form of double stranded DNA. Nevertheless, MGB compounds which would show preference to C-G (cytosine and guanine) rich regions are also theoretically possible. Therefore, oligonucleotides comprising a radical or moiety derived from minor groove binder molecules having preference for C-G regions are also within the scope of the present invention.

Some MGBs are capable of binding within the minor groove of double stranded DNA with an association constant of 10³M⁻¹ or greater. This type of binding can be detected by well established spectrophotometric methods such as ultraviolet (UV) and nuclear magnetic resonance (NMR) spectroscopy and also by gel electrophoresis. Shifts in UV spectra upon binding of a minor groove binder molecule and NMR spectroscopy utilizing the “Nuclear Overhauser” (NOSEY) effect are particularly well known and useful techniques for this purpose. Gel electrophoresis detects binding of an MGB to double stranded DNA or fragment thereof, because upon such binding the mobility of the double stranded DNA changes.

A variety of suitable minor groove binders have been described in the literature. See, for example, Kutyavin, et al. U.S. Pat. No. 5,801,155; Wemmer, D. E., and Dervan P. B., Current Opinion in Structural Biology, 7:355-361 (1997); Walker, W. L., Kopka, J. L. and Goodsell, D. S., Biopolymers, 44:323-334 (1997); Zimmer, C. & Wahnert, U. Prog. Biophys. Molec. Bio. 47:31-112 (1986) and Reddy, B. S. P., Dondhi, S. M., and Lown, J. W., Pharmacol. Therap., 84:1-111 (1999). In one group of embodiments, the MGB is selected from the group consisting of CC1065 analogs, lexitropsins, distamycin, netropsin, berenil, duocarmycin, pentamidine, 4,6-diamino-2-phenylindole and pyrrolo[2,1-c][1,4]benzodiazepines. A preferred MGB in accordance with the present disclosure is DPI₃ (see U.S. Pat. No. 6,727,356, the disclosure of which is incorporated herein by reference in its entirety).

Suitable methods for attaching MGBs through linkers to oligonucleotides or probes and have been described in, for example, U.S. Pat. Nos. 5,512,677; 5,419,966; 5,696,251; 5,585,481; 5,942,610; 5,736,626; 5,801,155 and 6,727,356. For example, MGB-oligonucleotide conjugates can be synthesized using automated oligonucleotide synthesis methods from solid supports having cleavable linkers. In other examples, MGB probes can be prepared from an MGB modified solid support substantially in accordance with the procedure of Lukhtanov et al. Bioconjugate Chem., 7: 564-567 (1996). (The disclosure of which is also incorporated herein by reference in its entirety.) According to these methods, one or more MGB moieties can be attached at the 5′-end, the 3′-end and/or at any internal portion of the oligonucleotide.

The location of an MGB moiety within an MGB-oligonucleotide conjugate can affect the discriminatory properties of such a conjugate. An unpaired region within a duplex will likely result in changes in the shape of the minor groove in the vicinity of the mismatched base(s). Since MGBs fit best within the minor groove of a perfectly-matched DNA duplex, mismatches resulting in shape changes in the minor groove would reduce binding strength of an MGB to a region containing a mismatch. Hence, the ability of an MGB to stabilize such a hybrid would be decreased, thereby increasing the ability of an MGB-oligonucleotide conjugate to discriminate a mismatch from a perfectly-matched duplex. On the other hand, if a mismatch lies outside of the region complementary to an MGB-oligonucleotide conjugate, discriminatory ability for unconjugated and MGB-conjugated oligonucleotides of equal length is expected to be approximately the same. Since the ability of an oligonucleotide probe to discriminate single base pair mismatches depends on its length, shorter oligonucleotides are more effective in discriminating mismatches. The first advantage of the use of MGB-oligonucleotides conjugates in this context lies in the fact that much shorter oligonucleotides compared to those used in the art (i.e., 20-mers or shorter), having greater discriminatory powers, can be used, due to the pronounced stabilizing effect of MGB conjugation. Consequently, larger delta Tms of allele-specific blocker probes can improve AS-PCR assay specificity and selectivity.

Blocker probes having MGB at the 5′ termini may have additional advantages over other blocker probes having a blocker moiety (e.g., MGB, PO₄, NH₂, PEG, or biotin) only at the 3′ terminus. This is at least because blocker probes having MGB at the 5′ terminus (in addition to a blocking moiety at the 3′-end that prevents extension) will not be cleaved during PCR amplification. Thus, the probe concentration can be maintained at a constant level throughout PCR, which may help maintain the effectiveness of blocking non-specific priming, thereby increasing cast-PCR assay specificity and selectivity.

In some embodiments, the allele-specific blocker probe can comprise one or more modified bases in addition to the naturally occurring bases adenine, cytosine, guanine, thymine and uracil. In some embodiments, the modified base(s) may increase the difference in the Tm between matched and mismatched target sequences and/or decrease mismatch priming efficiency, thereby improving not only assay specificity, bust also selectivity. In some embodiments of the methods and kits, the first allele-specific blocker probe binds to the same strand or sequence as the first allele-specific primer, while the second allele-specific blocker probe binds to the opposite strand and/or complementary sequence as the first allele-specific primer.

Modified bases are considered to be those that differ from the naturally-occurring bases by addition or deletion of one or more functional groups, differences in the heterocyclic ring structure (i.e., substitution of carbon for a heteroatom, or vice versa), and/or attachment of one or more linker arm structures to the base. Such modified base(s) may include, for example, 8-Aza-7-deaza-dA (ppA), 8-Aza-7-deaza-dG (ppG), locked nucleic acid (LNA) or 2′-O,4′-C-ethylene nucleic acid (ENA) bases. Other examples of modified bases include, but are not limited to, the general class of base analogues 7-deazapurines and their derivatives and pyrazolopyrimidines and their derivatives (described in PCT WO 90/14353; and U.S. application Ser. No. 09/054,630, the disclosures of each of which are incorporated herein by reference in their entireties). These base analogues, when present in an oligonucleotide, strengthen hybridization and improve mismatch discrimination. All tautomeric forms of naturally occurring bases, modified bases and base analogues may be included in the oligonucleotide primer and probes of the invention.

Similarly, modified sugars or sugar analogues can be present in one or more of the nucleotide subunits of an oligonucleotide conjugate in accordance with the invention. Sugar modifications include, but are not limited to, attachment of substituents to the 2′, 3′ and/or 4′ carbon atom of the sugar, different epimeric forms of the sugar, differences in the α- or β-configuration of the glycosidic bond, and other anomeric changes. Sugar moieties include, but are not limited to, pentose, deoxypentose, hexose, deoxyhexose, ribose, deoxyribose, glucose, arabinose, pentofuranose, xylose, lyxose, and cyclopentyl.

Modified internucleotide linkages can also be present in oligonucleotide conjugates of the invention. Such modified linkages include, but are not limited to, peptide, phosphate, phosphodiester, phosphotriester, alkylphosphate, alkanephosphonate, thiophosphate, phosphorothioate, phosphorodithioate, methylphosphonate, phosphoramidate, substituted phosphoramidate and the like. Several further modifications of bases, sugars and/or internucleotide linkages, that are compatible with their use in oligonucleotides serving as probes and/or primers, will be apparent to those of skill in the art. In addition, in some embodiments, the nucleotide units which are incorporated into the oligonucleotides of the MGB blocker probes of the present invention may have a cross-linking function (an alkylating agent) covalently bound to one or more of the bases, through a linking arm.

The “sugar” or glycoside portion of some embodiments of the MGB blocker probes of the present invention may comprise deoxyribose, ribose, 2-fiuororibose, 2-0 alkyl or alkenylribose where the alkyl group may have 1 to 6 carbons and the alkenyl group 2 to 6 carbons. In the naturally occurring nucleotides and in the herein described modifications and analogs the deoxyribose or ribose moiety forms a furanose ring. the glycosydic linkage is of the ˜configuration and the purine bases are attached to the sugar moiety via the 9-position. the pyrimidines via the I-position and the pyrazolopyrimidines via the I-position. The nucleotide units of the oligonucleotide are interconnected by a “phosphate” backbone, as is well known in the art. The oligonucleotide of the oligonucleotide-MGB conjugates (MGB blocker probes) of the present invention may include, in addition to the “natural” phosphodiester linkages, phosphorothiotes and methylphosphonates.

In some embodiments, detector probe is designed as short oligomers ranging from about 15-30 nucleotides, such as about 16, about 18, about 22, about 24, about 30, or any number in between. In some embodiments, the Tm of the detector probe ranges from about 60° C. to 70° C., about 61° C. to 69° C., about 62° C. to 68° C., about 63° C. to 67° C., or about 64° C. to 66° C., or any range in between. In some embodiments, the detector probe is a locus-specific detector probes (LST). In some other embodiments of the disclosed methods and kits, first and second locus-specific detector probes may comprise the same sequence or be the same sequence. In some embodiments the detector probe is a 5′ nuclease probe.

In some exemplary embodiments, the detector probe can comprises an MGB moiety, a reporter moiety (e.g., FAM™, TET™, JOE™, VIC™, or SYBR® Green), a quencher moiety (e.g., Black Hole Quencher™ or TAMRA™;), and/or a passive reference (e.g., ROX™). In some exemplary embodiments, the detector probe is designed according to the methods and principles described in U.S. Pat. No. 6,727,356. In some exemplary embodiments, the detector probe is a TaqMan® probe (Applied Biosystems, Foster City). In exemplary embodiments, the locus-specific detector probe can be designed according to the principles and methods described in U.S. Pat. No. 6,727,356. For example, fluorogenic probes can be prepared with a quencher at the 3′ terminus of a single DNA strand and a fluorophore at the 5′ terminus. In such an example, the 5′-nuclease activity of a Taq DNA polymerase can cleave the DNA strand, thereby separating the fluorophore from the quencher and releasing the fluorescent signal. In some embodiments, the detector probes are hybridized to the template strands during primer extension step of PCR amplification (e.g., at 60-65° C.). In yet other embodiments, an MGB is covalently attached to the quencher moiety of the locus-specific detector probes (e.g., through a linker). In some embodiments of the disclosed methods and kits, the first and second detector probes are the same and/or comprise the same sequence or are the same sequence.

In some embodiments, the locus-specific primer (LSP) is designed as a short oligomer ranging from about 15-30 nucleotides, such as about 16, about 18, about 22, about 24, about 30, or any number in between. In some embodiments, the Tm of the locus-specific primer ranges from about 60° C. to 70° C., about 61° C. to 69° C., about 62° C. to 68° C., about 63° C. to 67° C., or about 64° C. to 66° C., or any range in between.

Polymerase enzymes suitable for the practice of the present invention are well known in the art and can be derived from a number of sources. Thermostable polymerases may be obtained, for example, from a variety of thermophilic bacteria that are commercially available (for example, from American Type Culture Collection, Rockville, Md.) using methods that are well-known to one of ordinary skill in the art (see, e.g., U.S. Pat. No. 6,245,533). Bacterial cells may be grown according to standard microbiological techniques, using culture media and incubation conditions suitable for growing active cultures of the particular species that are well-known to one of ordinary skill in the art (See, e.g., Brock, T. D., and Freeze, H., J. Bacteriol. 98(1):289-297 (1969); Oshima, T., and Imahori, K, Int. J. Syst. Bacteriol. 24(1):102-112 (1974)). Suitable for use as sources of thermostable polymerases are the thermophilic bacteria Thermus aquaticus, Thermus thermophilus, Thermococcus litoralis, Pyrococcus furiosus, Pyrococcus woosii and other species of the Pyrococcus genus, Bacillus stearothermophilus, Sulfolobus acidocaldarius, Thermoplasma acidophilum, Thermus flavus, Thermus ruber, Thermus brockianus, Thermotoga neapolitana, Thermotoga maritima and other species of the Thermotoga genus, and Methanobacterium thermoautotrophicum, and mutants of each of these species. Preferable thermostable polymerases can include, but are not limited to, Taq DNA polymerase, Tne DNA polymerase, Tma DNA polymerase, or mutants, derivatives or fragments thereof.

Sources of nucleic acid samples in the disclosed compositions, methods and/or kits include, but are not limited to, human cells such as circulating blood, buccal epithelial cells, cultured cells and tumor cells. Also other mammalian tissue, blood and cultured cells are suitable sources of template nucleic acids. In addition, viruses, bacteriophage, bacteria, fungi and other micro-organisms can be the source of nucleic acid for analysis. The DNA may be genomic or it may be cloned in plasmids, bacteriophage, bacterial artificial chromosomes (BACs), yeast artificial chromosomes (YACs) or other vectors. RNA may be isolated directly from the relevant cells or it may be produced by in vitro priming from a suitable RNA promoter or by in vitro transcription. The present invention may be used for the detection of variation in genomic DNA whether human, animal or other. It finds particular use in the analysis of inherited or acquired diseases or disorders. A particular use is in the detection of inherited diseases.

In some embodiments, template sequence or nucleic acid sample can be gDNA. In other embodiments, the template sequence or nucleic acid sample can be cDNA. In yet other embodiments, as in the case of simultaneous analysis of gene expression by RT-PCR, the template sequence or nucleic acid sample can be RNA. The DNA or RNA template sequence or nucleic acid sample can be extracted from any type of tissue including, for example, formalin-fixed paraffin-embedded tumor specimens.

Any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

In the claims, any active verb (or its gerund) is intended to indicate the corresponding actual or attempted action, even if no actual action occurs. For example, the verb “hybridize” and gerund form “hybridizing” and the like refer to actual hybridization or to attempted hybridization by contacting nucleic acid sequences under conditions suitable for hybridization, even if no actual hybridization occurs. Similarly, “detecting” and “detection” when used in the claims refer to actual detection or to attempted detection, even if no target is actually detected.

Furthermore, it is to be understood that the inventions encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

The present disclosure provides the advantage that any of the combinations of listed improvements could be utilized by a skilled artisan in a particular situation. For example, the current invention can include a method or reaction mixture that employs improvements a, c, d and f; improvements b, c, and e; or improvements. It is to be understood that both the foregoing general description and the following detailed description are exemplary and explanatory only and are not restrictive of the invention, as claimed. The accompanying drawings, which are incorporated in and constitute a part of this specification, illustrate several exemplary embodiments of the disclosure and together with the description, serve to explain certain teachings.

All publications and patents cited in this specification are herein incorporated by reference as if each individual publication or patent were specifically and individually indicated to be incorporated by reference. Genbank records referenced by GID or accession number, particularly any polypeptide sequence, polynucleotide sequences or annotation thereof, are incorporated by reference herein. The citation of any publication is for its disclosure prior to the filing date and should not be construed as an admission that the present invention is not entitled to antedate such publication by virtue of prior invention.

Certain embodiments are further described in the following examples. These embodiments are provided as examples only and are not intended to limit the scope of the claims in any way.

### EXAMPLES

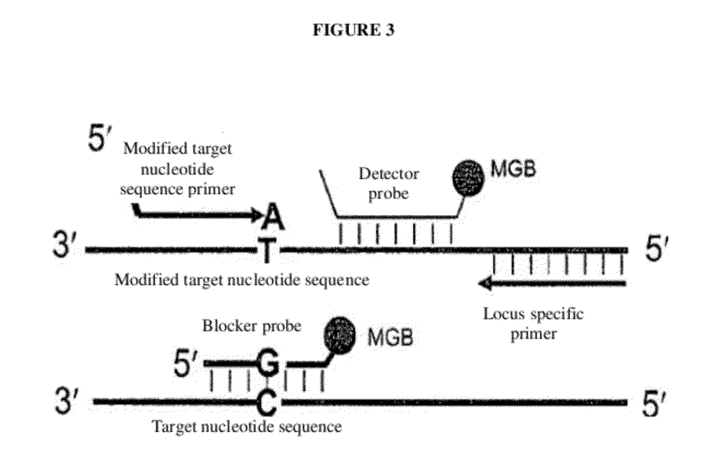

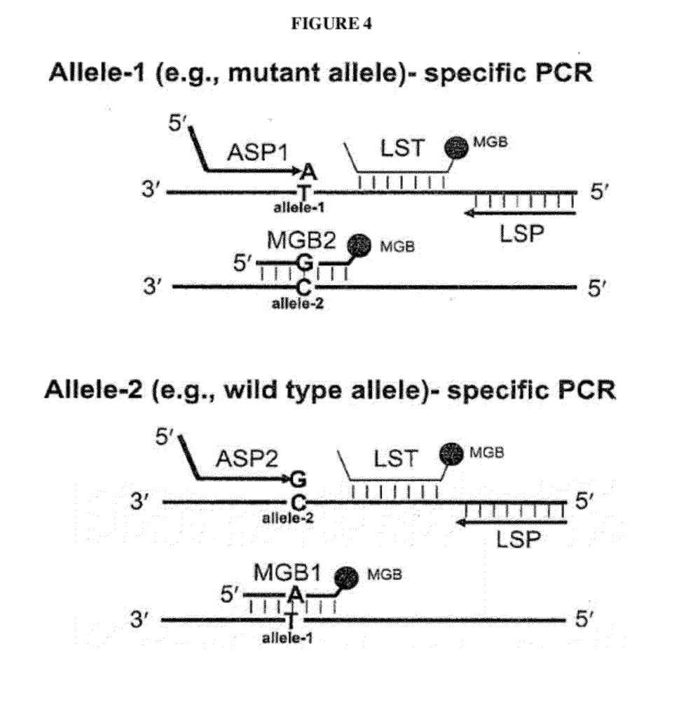

The methods described herein provide new methods for analyzing biological samples without pre-processing (or substantial pre-processing). As briefly mentioned above, potential target cells may be isolated from a biological sample that has not been substantially pre-processed, and then subjected to an assay for identifying allele-specific nucleotide sequences therein. Thus, these methods may comprise the steps of: 1) digital enrichment of target cells without substantially pre-processing the biological sample (e.g., unprocessed aliquots of the sample are prepared); and, 2) identification and enumeration of target cells present in the sample. The second step may be accomplished using any of the available cell and/or nucleic acid detection methods. As described above, this disclosure provides methods for identifying target cells from biological samples that have not been pre-processed (e.g., subjected to immuno-capture, density gradient and/or cell sorting enrichment procedures) (FIG. 2). The data presented here illustrates this method may be used to detect circulating tumor cells (CTCs) directly in unprocessed whole blood. The method includes digital enrichment of cells (FIG. 1) in an unprocessed whole blood and identification/enumeration of CTCs by detecting modified (e.g., mutated) target nucleic acid sequences expressed by such cells using castPCR (FIGS. 3, 4).

### Example 1

### Materials and Methods

The general schemes for digital enrichment of target cells combined with sensitive detection assays, such as castPCR, that are used in the following examples are illustrated in FIGS. 1-4. For each SNP that was analyzed, allele-specific primers were designed to target a first allele (i.e. allele-1) and a second allele (i.e. allele-2). The castPCR assay reaction mixture for allele-1 analysis included a tailed allele-1-specific primer (ASP1), one MGB allele-2 blocker probe (MGB2), one common locus-specific TaqMan probe (LST) and one common locus-specific primer (LSP). The castPCR assay reaction mixture for analysis of allele-2 included a tailed allele-2-specific primer (ASP2), one MGB allele-1 blocker probe (MGB1), one common locus-specific TaqMan probe (LST) and one common locus-specific primer (LSP). All reactions were carried out essentially as described in US 2010/0221717 A1 (U.S. Ser. No. 12/641,321) and US 2010/0285478 A1 (U.S. Ser. No. 12/748,329). Additional details are described below.

### Example 2

### Detection of Model CTCs Spiked in to Whole Blood

As a proof of concept experiment, a small number of cells of a cancer cell line with a known genetic phenotype (e.g., a known mutation) were added into normal whole blood samples known not to contain mutated cells (e.g., the blood was “spiked” with model CTCs). The data indicate that RT castPCR may be used to detect and quantitate rare target cells in a biological sample.

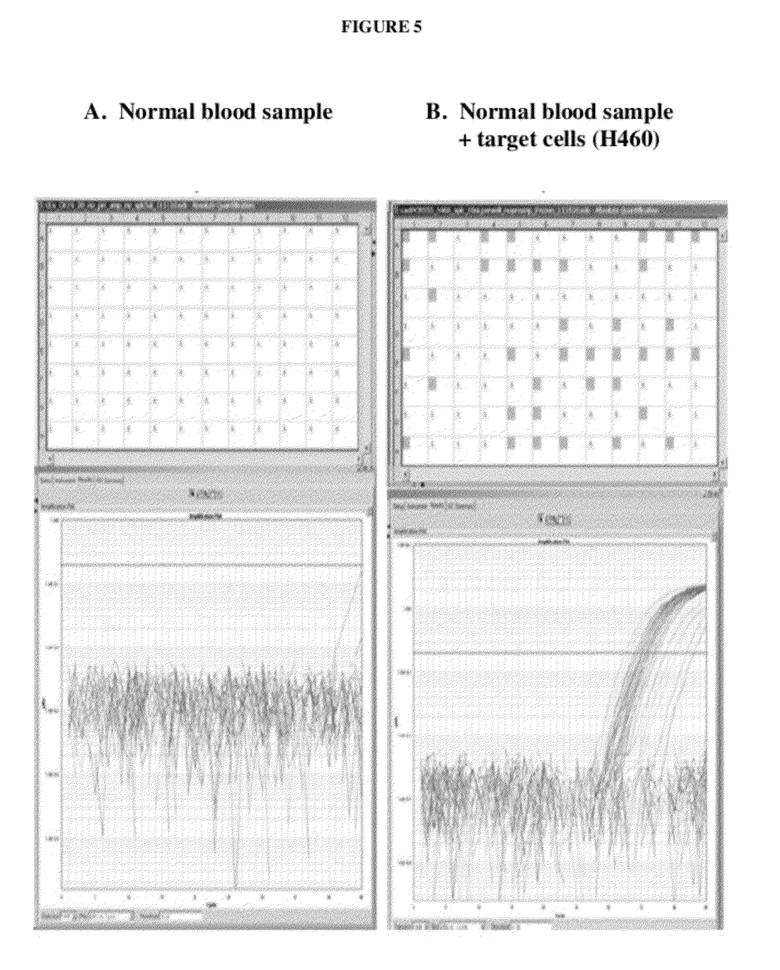

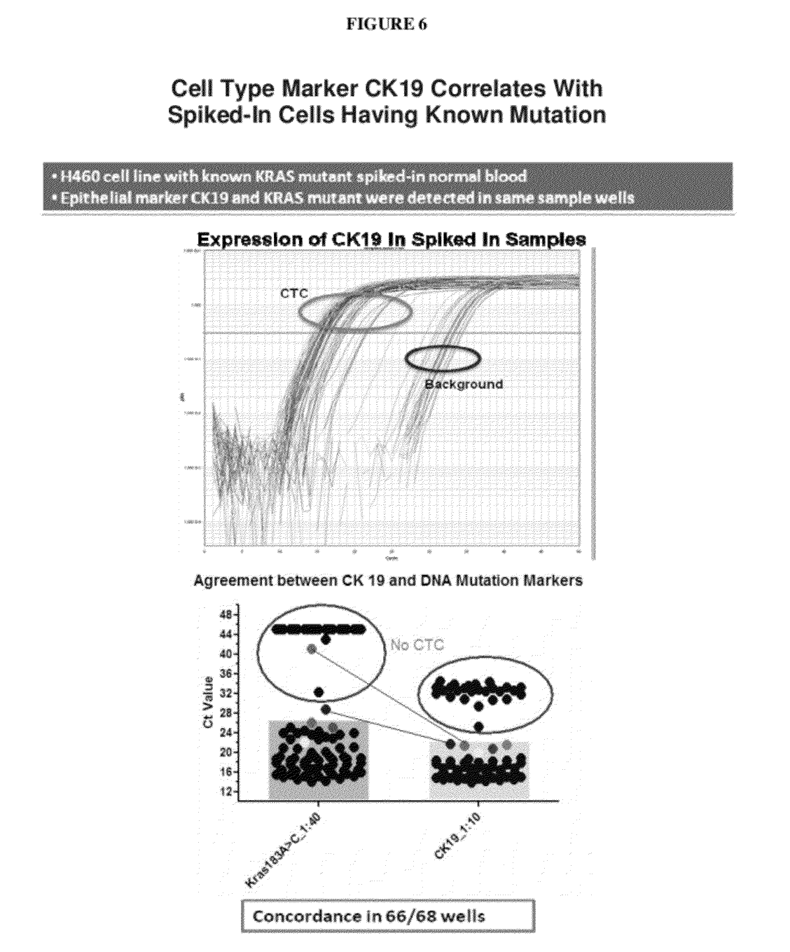

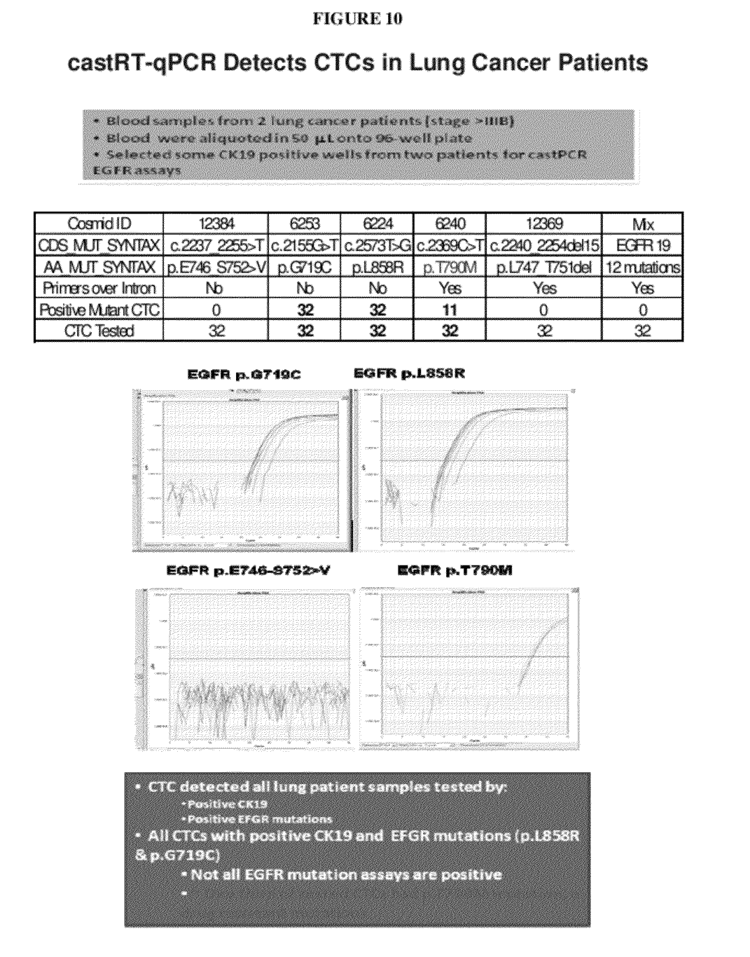

The H460 lung cancer cell line is known to contain the KRAS mutation p.Q61H c.183A>T (castPCR Assay ID 555) (and to express the CK19 epithelial cell marker). To test the new methods described herein, H460 cells were “spiked” into normal blood samples. An estimated 25-50 cells (e.g., about 38 cells estimated) as quantitated using the Auto Cell Counter (Invitrogen) were added into 1 ml normal whole blood followed by distribution of 2.5 ul-50 ul aliquots into separate wells of a 96-well plate. Each well was estimated to contain a single model CTC (e.g., H460) “target” cell in a background of non-target cells (e.g., 10-400×10³ cells/well). Total RNA was extracted by MagMAX-96 Blood RNA Isolation Kit from all the cells in each well according to the manufacturer's protocol. RNAs were reverse transcribed into cDNA using random primers. The extracted RNA was either directly used for mutation detection by castPCR (e.g., RT castPCR) as in FIGS. 5A and 5B, or pre-amplified first by target specific primer sets which can be the same as the castPCR primers. For example, in FIGS. 6 and 10, target cells were identified by the expression of epithelial cell markers such as CK19 and allelic mutations in genes such as KRAS or EGFR by first pre-amplifying samples in the same wells prior to detection analysis. Thus, in some embodiments, sample pre-amplification can be employed for multiple target detections (e.g., multiplexing different nucleic acid sequences within the same sample well). Quantification of target cells was performed by detecting CK19 expression and/or the KRAS mutation using RT castPCR. The number of CTCs in a blood sample can be expressed as CTC number per mL whole blood or per sample in a certain blood volume.

The results of these experiments are illustrated by FIGS. 5A and 5B and 6. The samples were distributed into the wells of a 96-well plate, with each well containing 5 uL blood (with or without “spiked-in” target cells). The spiked cell lines (H460) purchased from AACC contains known KRAS mutant p.Q61H (Cast-PCR assay No. 555, Kras183A>C). A control normal blood sample into which target cells were not added was included (FIG. 5A). RNA was extracted from each sample and castPCR was used to identify which sample wells, without sample pre-amplification, contain target cells. There was no detectable expression of KRAS p.Q61H in control samples without cell spiking-in (FIG. 5A). There were 38 sample wells containing the pQ61H mutation, which was very close to the number of estimated spiked cells. The results clearly show that castPCR can detect a single nucleic acid copy in a sample well containing a single (estimated) target cells in a mixed sample of whole blood (e.g., 5 μL blood or 10-400×10³ total cells). Importantly, the KRAS mutation was not identified in plates containing only normal blood cells (e.g., without being spiked with target cells; FIG. 5A), while 38 positive aliquots wells were identified from the samples spiked with target cells (FIG. 5B).

FIG. 6 illustrates data from an experiment showing that CK19 expression may be detected along with a KRAS mutation in samples with pre-amplification. CK19 expression was detected by custom-designed TaqMan gene expression assays (Applied Biosystems), with the following oligonucleotides: Forward primer CGACTACAGCCACTACTACACGA (SEQ ID NO.: 1); Reverse primer AGCCTGTTCCGTCTCAAACTTGGT (SEQ ID NO.: 2), and TaqMan probe (FAM)TCCTGCAGATCGACAATGC(MGB) (SEQ ID NO.: 3). H460 cells were “spiked” into normal blood samples as described above for FIG. 5. As shown therein, CK19 expression was detected in all samples comprising spiked-in cells (FIG. 6, top panel). Importantly, the samples expressing high CK19 were also determined to contain the KRAS 183A>C mutation (the H460 cell line mutation) (FIG. 6, bottom panel), while those samples expressing low CK19 level were determined not to contain that mutation (or it was only detected at late Ct values). If the “cut-off” value for CK19 was set at a Cq value of 21, and that for KRAS 183A>C mutation set at 25, only two wells were observed to have discordant CK 19 and KRAS values (FIG. 6, bottom). These results indicate that the combined methods described herein may be used to detect extremely low numbers of cells in blood samples that have not been pre-processed (e.g., subjected to immuno-capture, density gradient and/or cell sorting enrichment procedures).

Furthermore, wild type KRAS expression (Assay 555 WT), which expression is expected at lower level in normal cells (especially blood proliferative lymphocytes), can be detected in both positive and negative wells of CK19 and KRAS mutants. On the other hand, an assay configured to detect a KRAS mutation not present in the H460 cell line (Assay ID 522) did not detect any positive cells in any of the wells (data not shown). These results further confirm the detection of rare target cells in whole blood without biochemical or physical pre-processing.

### Example 3

### Detection of KRAS and EGFR Mutations in Cells from Spiked-in Blood Samples

In this example, H460 or H1975 cells were “spiked” into normal blood samples as described above for Example 2. Briefly, an estimated 20-60 cells (as indicated—see FIG. 10, column 4) as quantitated using the Auto Cell Counter (Invitrogen) were added into 1 ml normal whole blood followed by distribution of 5 or 10 μL aliquots (as indicated—see FIG. 10, column 2) were added to separate wells of a 96-well plate. Each well was estimated to contain a single model CTC (e.g., H460 or H1975) “target” cell in a background of non-target cells (e.g., 50-100×10³ white blood cells/well). Total RNA was extracted by MagMAX-96 Blood RNA Isolation Kit from all the cells in each well according to the manufacturer's protocol. RNAs were reverse transcribed into cDNA using random primers. The extracted RNA was then directly used for mutation detection by castPCR.

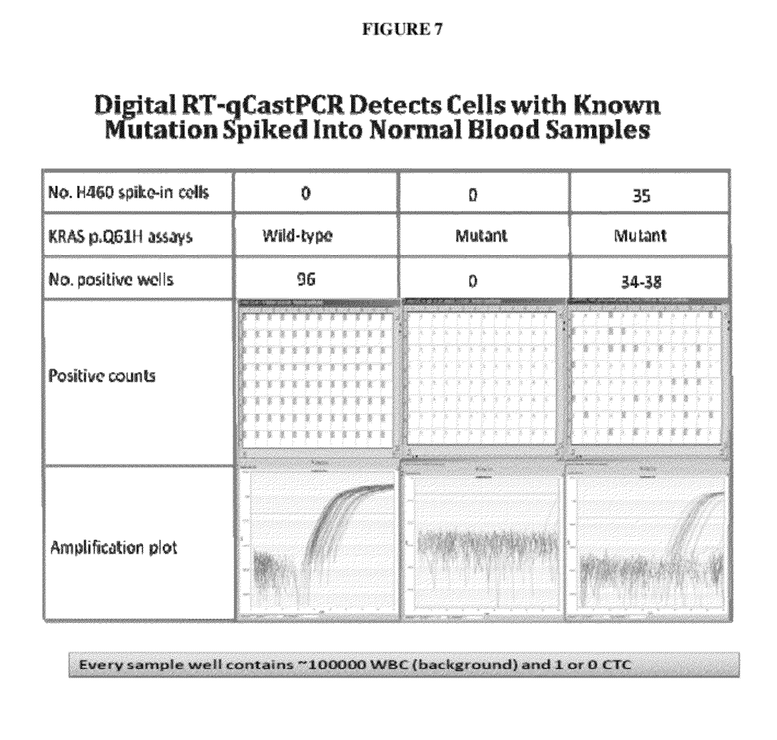

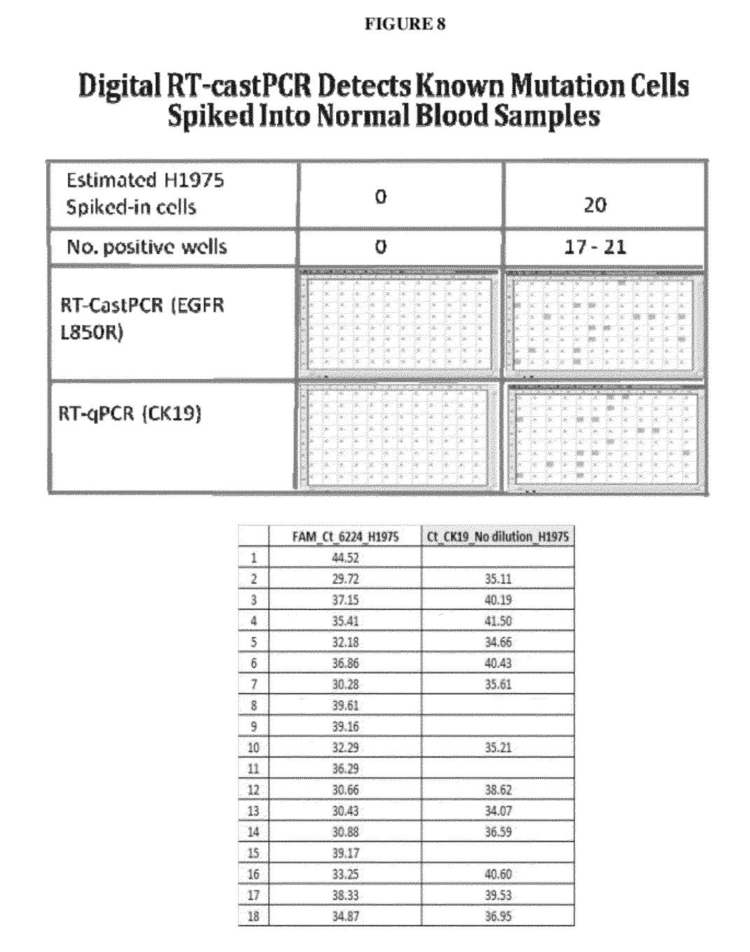

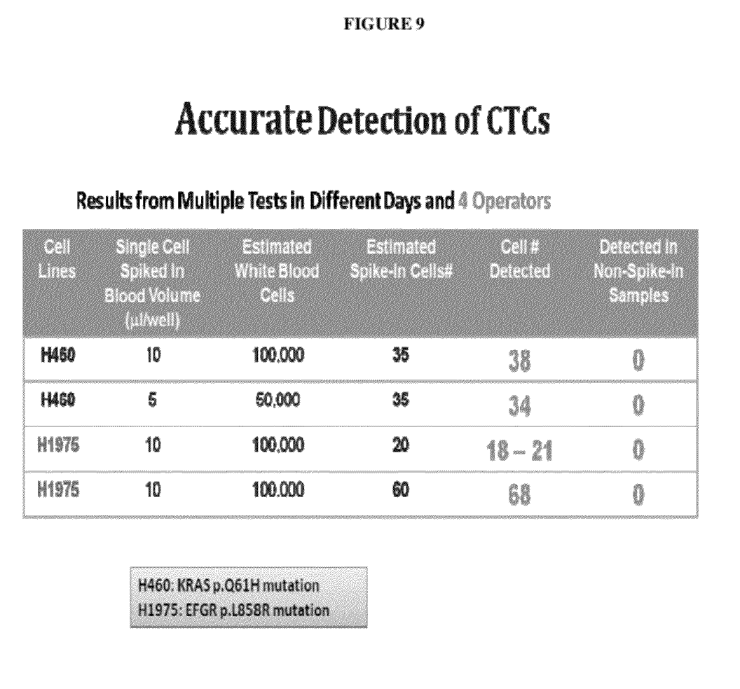

The results of these experiments are illustrated by FIGS. 7-10. The samples were distributed into the wells of a 96-well plate, with each well containing 5 or 10 μL blood (with or without “spiked-in” target cells). Normal blood samples into which target cells were not added were included as controls (FIG. 7, column 3 and FIG. 8, column 2). RNA was extracted from each sample and castPCR was used to identify which sample wells, without sample pre-amplification, contained target cells.

With regard to the experiments using H460 spiked-in cells (FIG. 7): There was detectable expression of wild type KRAS in all sample wells (FIG. 7, column 2). There was no detectable expression of KRAS p.Q61H in control sample wells without cell spiking-in (FIG. 7, column 3). There were 34-38 sample wells containing the pQ61H mutation, which was very close to the number of estimated spiked cells (FIG. 7, column 4).

With regard to the experiments using H1975 spiked-in cells (FIG. 8): There was no detectable expression of EGFR p.L850R in control sample wells without cell spiking-in (FIG. 8, column 2). There were 17-21 sample wells containing the p.L850R mutation, which was very close to the number of estimated spiked cells (FIG. 8, column 3).

The results of these experiments are also illustrated by FIG. 10. which shows that there was no detectable expression of KRAS or EGFR mutants in control sample wells without cell spiking-in (FIG. 10, column 6) and that there were positive sample wells containing the spiked-in cells having KRAS or EGFR mutations (p.Q61H or p.L858R mutations, respectively), which were very close to the number of estimated spiked cells (FIG. 10, columns 4 and 5). The results clearly show that castPCR can detect a single nucleic acid copy in a sample well containing a single (estimated) target cell in a mixed sample of whole blood.

FIG. 8 further illustrates data from an experiment showing that CK19 expression may be detected along with an EGFR mutation in spiked-in samples. CK19 expression was detected by custom-designed TaqMan gene expression as described in Example 2. As shown, CK19 expression correlated well with samples also showing positive for the EGFR mutation (FIG. 8, top panel, column 3). The Ct values from the castPCR analysis are shown (FIG. 8, bottom panel). Collectively, these results indicate that the combined methods described herein may be used to detect extremely low numbers of cells in blood samples that have not been pre-processed (e.g., subjected to immuno-capture, density gradient and/or cell sorting enrichment procedures).

Furthermore, wild type KRAS expression (Assay 555 WT), which expression is expected at lower level in normal cells (especially blood proliferative lymphocytes), can be detected in both positive and negative wells of CK19 and KRAS mutants. On the other hand, an assay configured to detect a KRAS mutation not present in the H460 cell line (Assay ID 522) did not detect any positive cells in any of the wells (data not shown). These results further confirm the detection of rare target cells in whole blood without biochemical or physical pre-processing.

### Example 4

### Detection of KRAS and EGFR Mutations in Whole Blood from Lung Cancer Patients

Two blood samples from lung cancers with stage IIIB and IV, respectively, were tested using essentially the procedures described in Example 2 except that the samples were not spiked, and castPCR assays using different primer/probe oligonucleotides were performed. Fifty μL aliquots of unprocessed, whole blood were distributed among the wells of a 96-well plate. RNA extraction, reverse-transcription and target specific pre-amplification were performed as previously described. Six castPCR assays were selected for screening EGFR mutations of CK-19 positive cells (FIG. 7).

All wells in both blood samples scored positive for CK19 expression, indicating there were likely more than 100 CTCs in 5 mL whole blood. castPCR assays were then performed to detect EGFR mutations in 16 CK19-positive wells (or 16 different cells) from both patient samples (FIG. 6). Interestingly, mutation p.L858R, one of the most common EGFR mutations in lung cancers, and mutation p.G719C, were positive for all 32 cells (16 positive wells from each sample) tested. Some CK19 positive cells (11 out of 32) tested positive for mutation T790M with high Ct values, probably due to the design of reverse primer at 5′ end (e.g., including five nucleotides in an intron, which reduced PCR efficiency). T790M is one of the CTC mutation markers indicating drug resistance to EGFR inhibitors. On the other hand, mutation EGFR p.E746-S752>V was not detected in any of the CK19-positive cells. All sample wells of either positive or negative EGFR mutations had strong expression of corresponding wild type EGFRs (FIG. 8). The EGFR mutation profiles were almost identical in both patient samples.

### Example 5

### CTC Detection in Whole Blood from Lung Cancer Patients

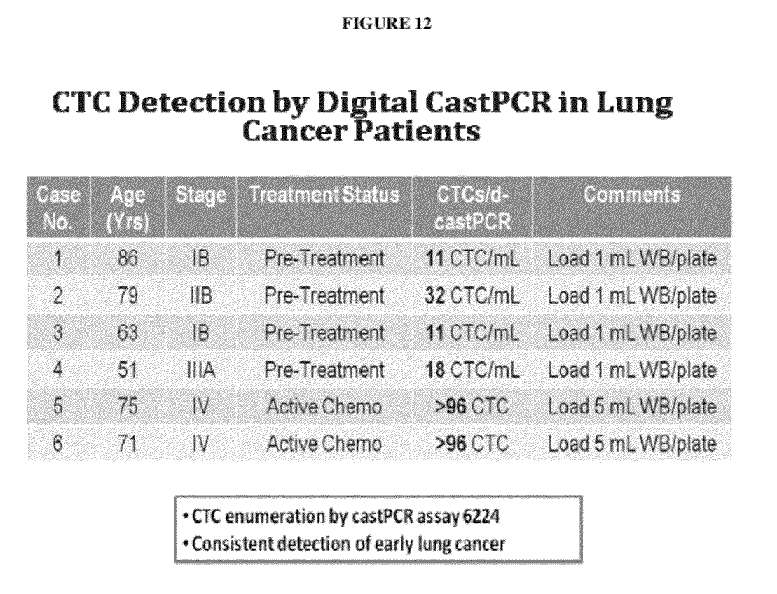

Blood samples from patients of different ages having lung cancers at various stages and having different treatment status (FIG. 12, columns 2-4) were analyzed using the methods described herein.

Briefly, blood samples from lung cancers were tested using essentially the procedures described in Example 4 (using 1 ml whole blood per 96-well plate). All aliquots tested (10 μL/well) scored positive for EGFR mutation expression (indicative of samples that are positive for CTCs). For example, as few as 11 CTCs were detected in 1 ml samples from early stage cancer patients while more than 96 CTCs were detected in 1 ml samples from late stage cancer patients undergoing active chemotherapy (FIG. 12, column 5). These data suggest that the methods described herein can be used for detection of early stage cancers (when CTCs are typically present at very low numbers) as well as late stage cancers (when CTCs are typically more abundant).

In summary, the experimental evidence described above demonstrates that the combination of “digital enrichment” and transcriptional mutation analysis using highly sensitive detection methods, such as castPCR, provides direct enumeration and molecular characterization of rare cells (e.g., CTCs) without pre-processing biological samples (e.g., without processing whole blood) prior to analysis.

What is claimed is:

1. A method for detecting a target cell present in a biological sample that has not been substantially pre-processed, the method comprising the steps of, in combination:
a) preparing aliquots of the biological sample such that each aliquot contains or does not contain a single target cell; and,
b) assaying the aliquots to detect a target cell therein.

2. The method of claim 1 wherein the target cell has at least one first allele of interest not present in a non-target cell, and step b) is performed by:
1) forming a reaction mixture by combining:
i) a nucleic acid sample representative of the biological sample;
ii) a first allele-specific primer being complementary to a second allele of interest except that the 3′ terminal nucleotide of the primer is complementary to the first allele of interest and not the second allele of interest;
iii) a first blocker probe being complementary to the target nucleotide sequence, lacking complete complementarity to the first allele of interest, and comprising a 3′ non-extendable blocking moiety;
iv) a first locus-specific primer complementary to the nucleic acid sample at a region therein which is 3′ from and on the opposite strand to that which the first allele-specific primer is complementary; and,
v) a first detector probe complementary to a region of the target nucleotide sequence between that which the first allele-specific primer and the first locus-specific primer are complementary;
2) carrying out an amplification reaction on the reaction mixture using the first allele-specific primer and the first locus-specific primer to form an amplicon; and,
3) detecting the amplicon by detecting the first detector probe.

3. The method of claim 2, further comprising:
1) forming a second reaction mixture by combining:
i) a nucleic acid sample representative of the biological sample in claim 1;
ii) a third allele-specific primer being complementary to a fourth allele of interest except that the 3′ terminal nucleotide of the primer is complementary to a third allele of interest and not the fourth allele of interest;
iii) a blocker probe being complementary to the target nucleotide sequence, lacking complete complementarity to the third allele of interest, and comprising a 3′ non-extendable blocking moiety;
iv) a second locus-specific primer complementary to the nucleic acid sample at a region therein which is 3′ from and on the opposite strand to that which the third allele-specific primer is complementary; and,
v) a second detector probe complementary to a region of the target nucleotide sequence between that which the third allele-specific primer and the second locus-specific primer are complementary;
2) carrying out an amplification reaction on the reaction mixture using the third allele-specific primer and the second locus-specific primer to form an amplicon; and,
3) detecting the amplicon by detecting the detector probe.

4. The method of claim 2 or 3, further comprising quantitating the amplicon.

5. The method of claim 4, further comprising comparing the change in a detectable property of the first detector probe in the first reaction mixture to the change in a detectable property of the second detector probe in the second reaction mixture.

6. A method for detecting at least a first allele of interest in target cell present within a biological sample that has not been substantially pre-processed, the method comprising the steps of, in combination:
i) preparing aliquots of the biological sample such that each aliquot contains about one to five target cells; and,
ii) assaying the aliquot to detect the target cells therein, wherein the target cell has at least one first allele of interest by:
1) forming a reaction mixture by combining:
i) a nucleic acid sample from said biological sample;
ii) a first allele-specific primer being complementary to a second allele of interest except that the 3′ terminal nucleotide of the primer is complementary to the first allele of interest and not the second allele of interest;
iii) a first blocker probe being complementary to the target nucleotide sequence, lacking complete complementarity to the first allele of interest, and comprising a 3′ non-extendable blocking moiety;
iv) a first locus-specific primer complementary to the nucleic acid sample at a region therein which is 3′ from and on the opposite strand to that which the first allele-specific primer is complementary; and,
v) a first detector probe complementary to a region of the target nucleotide sequence between that which the first allele-specific primer and the first locus-specific primer are complementary;
2) carrying out an amplification reaction on the reaction mixture using the first allele-specific primer and the first locus-specific primer to form an amplicon; and,
3) detecting the amplicon by detecting the first detector probe.

7. The method of claim 6, further comprising:
c) forming a second reaction mixture by combining:
1) a nucleic acid sample from the biological sample of part 1 of claim 6;
2) a third allele-specific primer being complementary to a fourth allele of interest except that the 3′ terminal nucleotide of the primer is complementary to a third allele of interest and not the fourth allele of interest;
3) a blocker probe being complementary to the target nucleotide sequence, lacking complete complementarity to the third allele of interest, and comprising a 3′ non-extendable blocking moiety;
4) a second locus-specific primer complementary to the nucleic acid sample at a region therein which is 3′ from and on the opposite strand to that which the third allele-specific primer is complementary; and,
5) a second detector probe complementary to a region of the target nucleotide sequence between that which the third allele-specific primer and the second locus-specific primer are complementary;
d) carrying out an amplification reaction on the reaction mixture using the third allele-specific primer and the second locus-specific primer to form an amplicon; and,
e) detecting the amplicon by detecting the detector probe.

8. The method of claim 6 or 7, further comprising quantitating the amplicon.

9. The method of claim 8, further comprising comparing the change in a detectable property of the first detector probe in the first reaction mixture to the change in a detectable property of the second detector probe in the second reaction mixture.

10. The method of any one of claims 1-9 wherein the target cell is detected by assaying the DNA of the target cell.

11. The method of any one of claims 1-9 wherein the target cell is detected by assaying the RNA of the target cell.

12. The method of any one of claims 1-11 wherein each at least one aliquot contains a single target cell.

13. The method of any one of claims 1-12 wherein each aliquot contains either zero target cells or a single target cell.

14. The method of claim 1, wherein said assaying employs the identification/detection of any one or more useful allele-specific biomarkers.

15. The method of claim 1 or 14, wherein said assaying employs the identification/detection of any one or more useful cell type-specific markers.

16. The method of claim 14, wherein said identification/detection of any one or more allele-specific biomarkers is performed by a molecular-based method.

17. The method of claim 16, wherein said molecular-based method is selected from the group consisting of allele-specific PCR (AS-PCR), cast-PCR, targeted HTP-sequencing, or proximity ligation assay (PLA).

18. The method of claim 15, wherein said cell type-specific markers are selected from the group consisting of cytokeratin, CK-19, EPCAM, ICAM, or CEA.

19. The method of claim 16, wherein said molecular-based method is capable of identifying allelic variants selected from the group consisting of BRAF-1799TA, CTNNB1-121AG, CTNNB1-134CT, EGFR-2369CT, EGFR-2573TG, KRAS-34GA, KRAS-35GA, KRAS-38GA, KRAS-176CG, KRAS-183AC, NRAS-35GA, NRAS-38GA, NRAS-181CA, NRAS-183AT, TP53-524GA, TP53-637CT, TP53-721TG, TP53-733GA, TP53-742CT, TP53-743GA, TP53-817CT, or those as described in, for example, US 2010/0221717 A1 (U.S. Ser. No. 12/641,321) and US 2010/0285478 A1 (U.S. Ser. No. 12/748,329)


SEQUENCE LISTING (3 sequences)

SEQ ID NO: 1 - DNA, 23 bp, Artificial Sequence
  Description of Artificial Sequence Synthetic primer
cgactacagccactactacacga

SEQ ID NO: 2 - DNA, 24 bp, Artificial Sequence
  Description of Artificial Sequence Synthetic primer
agcctgttccgtctcaaacttggt

SEQ ID NO: 3 - DNA, 19 bp, Artificial Sequence
  Description of Artificial Sequence Synthetic probe
tcctgcagatcgacaatgc